(19) United States
(12) Patent Application Publication
(10) Pub. No.: US 2025/0195244 A1
(43) Pub. Date: Jun. 19, 2025

(54) CIRCULATION MANAGEMENT THROUGH BLOOD VESSEL REMODELING

(71) Applicant: EDWARDS LIFESCIENCES CORPORATION, Irvine, CA (US)

(72) Inventors: David Maimon, Atlit (IL); Michael G. Valdez, Riverside, CA (US)

(73) Assignee: EDWARDS LIFESCIENCES CORPORATION, Irvine, CA (US)

(21) Appl. No.: 19/070,358

(22) Filed: Mar. 4, 2025

Related U.S. Application Data
(63) Continuation of application No. PCT/US2023/075871, filed on Oct. 3, 2023.
(60) Provisional application No. 63/513,533, filed on Jul. 13, 2023.
(60) Provisional application No. 63/482,196, filed on Jan. 30, 2023.
(60) Provisional application No. 63/378,465, filed on Oct. 5, 2022.

(51) Int. Cl. A61F 2/82

(52) U.S. Cl. CPC A61F 2/82; A61F 2210/0014; A61F 2220/0008; A61F 2250/0004

(57) ABSTRACT

A method of increasing compliance in a blood vessel involves accessing a target site in a blood vessel using a transcatheter access path, deploying a stent at the target site in the blood vessel, reshaping the blood vessel to an axially-bent configuration of the blood vessel by causing the stent to assume a biased, axially-bent configuration of the stent within the blood vessel, thereby pushing blood through the blood vessel, and reshaping the blood vessel to a relatively-straight configuration of the blood vessel using pressure forces of blood disposed within the blood vessel.

### RELATED APPLICATION(S)

This application is a continuation of International Patent Application No. PCT/US23/75871, filed Oct. 3, 2023, which claims the benefit of U.S. Provisional Patent Application Ser. Nos. 63/513,533, filed Jul. 13, 2023, 63/482,196, filed Jan. 30, 2023, and 63/378,465, filed Oct. 5, 2022, the complete disclosures of which are hereby incorporated by reference in their entirety.

### BACKGROUND

The present disclosure generally relates to the field of medical implant devices. Insufficient or reduced compliance in certain blood vessels, including arteries such as the aorta, can result in reduced perfusion, cardiac output, and other health complications. Restoring compliance and/or otherwise controlling flow/circulation in such blood vessels can improve patient outcomes.

### SUMMARY

Described herein are devices, methods, and systems that improve circulation in a blood vessels, such as blood vessels that are undesirably stiff and/or non-compliant. Devices associated with the various examples of the present disclosure can include stents designed to cyclically reshape a target blood vessel segment in a manner as to affect blood flow therein. Such stents, and/or portions thereof, can be configured to transition between axially bent and straight shapes for the purpose of reducing systolic pressure and/or pushing blood flow through the target vessel during diastole. Such stents can further be configured to transition between non-circular and circular cross-sectional shapes as the stent transitions between axially bent and straight configurations, which can further improve flow in the target vessel.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loudspeakers, headphones, pressure transducers, temperature transducers, or using any combination of suitable technologies.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIGS. 1A, 1B-1, and 1B-2 illustrates example cardiac and vascular anatomy of a patient, including details of example compliant and non-compliant aortas.

FIGS. 2A-1 and 2B-1 show side and axial cross-sectional views, respectively, of a healthy blood vessel experiencing compliant expansion.

FIGS. 2A-2 and 2B-2 show side and axial cross-sectional views, respectively, of a healthy blood vessel experiencing compliant contraction.

FIGS. 3A and 3B show side and axial cross-sectional views, respectively, of a stiff blood vessel experiencing compromised expansion.

FIG. 4 shows a side view of a flow-leveling stent in a relaxed, crimped/bent configuration in accordance with one or more examples.

FIGS. 5A and 5B show side cutaway and axial cross-sectional views, respectively, of a flow-leveling stent implanted in a segment of an aorta of a patient, the flow-leveling stent being configured in a crimped/bent configuration in accordance with one or more examples.

FIGS. 6A and 6B show side cutaway and axial cross-sectional views, respectively, of the flow-leveling stent of FIG. 5A implanted in a segment of an aorta of a patient, the flow-leveling stent being configured in a straightened configuration in accordance with one or more examples.

FIGS. 7-1 and 7-2 show an arch-clamp implant device implanted in an aortic arch, with the arch-clamp device shown in crimped and expanded configurations, respectively, in accordance with one or more examples.

FIG. 8 shows a perspective view of an arch-clamp implant device in accordance with one or more examples.

FIGS. 9-1, 9-2, and 9-3 illustrate a flow diagram for a process for implanting a flow-leveling arch-clamp device in an aortic arch in accordance with one or more examples.

FIGS. 10-1, 10-2, and 10-3 provide images of the stent and certain anatomy corresponding to operations of the process of FIGS. 9-1, 9-2, and 9-3 according to one or more examples.

FIGS. 11A and 11B show views of a flow-leveling stent assembly including a connecting arm in a relaxed, crimped/bent configuration in accordance with one or more examples.

FIGS. 12A and 12B show side cutaway and axial cross-sectional views, respectively, of a blood vessel segment spanned by an implanted flow-leveling stent assembly in accordance with one or more examples.

FIGS. 13A and 13B show side cutaway and axial cross-sectional views, respectively, of the flow-leveling stent assembly of FIG. 12A implanted in a blood vessel segment of a patient, the flow-leveling stent assembly being configured in a straightened configuration in accordance with one or more examples.

FIG. 14 shows a flow-leveling implant device in a bent configuration in accordance with one or more examples.

FIG. 15 shows a flow-leveling implant device in a bent configuration in accordance with one or more examples.

FIG. 16 shows a flow-leveling implant comprising a contact pad, the implant being configured in a bent configuration in accordance with one or more examples.

FIG. 17 shows a flow-leveling implant comprising a coil/spring connecting arm configured in a bent configuration in accordance with one or more examples.

FIG. 18 shows a flow-leveling stent assembly including one or more tapered stent anchors connected by a connecting arm in accordance with one or more examples.

FIGS. 19A and 19B show side cutaway and axial cross-sectional views, respectively, of a blood vessel segment spanned by an implanted flow-leveling stent assembly in accordance with one or more examples.

FIGS. 20A and 20B show side cutaway and axial cross-sectional views, respectively, of the flow-leveling stent assembly of FIG. 19A implanted in a blood vessel segment of a patient, the flow-leveling stent assembly being configured in a straightened configuration in accordance with one or more examples.

FIGS. 21A and 21B show views of a flow-leveling stent including ovalizing arms in accordance with one or more examples.

FIGS. 22A and 22B show side cutaway and axial cross-sectional views, respectively, of a flow-leveling stent including ovalizing arms implanted in a blood vessel segment in accordance with one or more examples.

FIGS. 23A and 23B show side cutaway and axial cross-sectional views, respectively, of a flow-leveling stent including ovalizing arms implanted in a blood vessel segment of a patient, the flow-leveling stent being configured with the arms thereof in a straightened configuration in accordance with one or more examples.

FIGS. 24A and 24B show side cutaway and axial cross-sectional views, respectively, of a flow-leveling stent including joined ovalizing arms implanted in a blood vessel segment in accordance with one or more examples.

FIGS. 25A and 25B show side cutaway and axial cross-sectional views, respectively, of a flow-leveling stent including joined ovalizing arms implanted in a blood vessel segment of a patient, the flow-leveling stent being configured with the arms thereof in a straightened configuration in accordance with one or more examples.

FIGS. 26A and 26B show side cutaway and axial cross-sectional views, respectively, of a flow-leveling stent assembly including stent segments coupled by ovalizing arms implanted in a blood vessel segment in accordance with one or more examples.

FIGS. 27A and 27B show side cutaway and axial cross-sectional views, respectively, of a flow-leveling stent assembly including stent segments coupled by ovalizing arms implanted in a blood vessel segment of a patient, the flow-leveling stent assembly being configured with the arms thereof in a straightened configuration in accordance with one or more examples.

FIGS. 28A and 28B show views of a flow-leveling stent including vessel re-shaping/ovalizing arms in accordance with one or more examples.

FIG. 29 shows a blood-vessel-reshaping implant device with at least a portion of an anchor/stent and/or reshaping arm thereof covered with a covering in accordance with one or more examples.

FIG. 30A shows a side cutaway of a flow-leveling stent implanted in an example blood vessel segment in accordance with one or more examples.

FIGS. 30B, 30C, and 30D show axial cross-sectional views of the implanted flow-leveling stent of FIG. 30A in accordance with one or more examples.

FIGS. 31A and 31B show side cutaway and axial cross-sectional views, respectively, of a flow-leveling implant with straightened arms in a high-pressure condition in accordance with one or more examples.

FIG. 32 shows a side view of a blood vessel reshaping implant assembly/device comprising a plurality of anchors coupled by vessel reshaping arm(s) in accordance with one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top.” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### Vascular Anatomy and Compliance

Certain examples are disclosed herein in the context of vascular implant devices, and in particular, flow-leveling and/or compliance-enhancing stent implant devices implanted/implantable in the aorta. However, although certain principles disclosed herein may be particularly applicable to the anatomy of the aorta, it should be understood that implant devices in accordance with the present disclosure may be implanted in, or configured for implantation in, any suitable or desirable blood vessels or other anatomy, such as the inferior vena cava.

The anatomy of the heart and vascular system is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles may be prompted by signals generated by the electrical system of the heart.

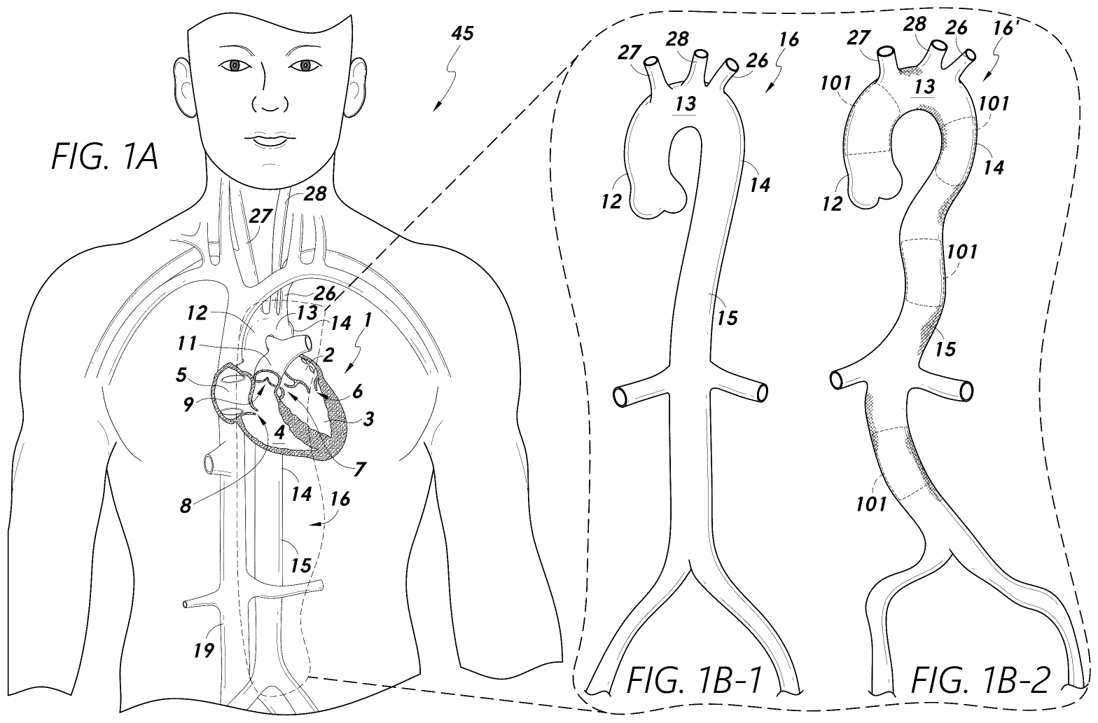

FIG. 1A illustrates an example representation of a heart 1 and associated vasculature having various features relevant to one or more examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery 11 via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left and right pulmonary arteries that branch off of the pulmonary trunk, as shown.

The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage.

The vasculature of the human body, which may be referred to as the circulatory system, cardiovascular system, or vascular system, contains a complex network of blood vessels with various structures and functions and includes various veins (venous system) and arteries (arterial system). Generally, arteries, such as the aorta 16, carry blood away from the heart, whereas veins, such as the inferior and superior vena cavae, carry blood back to the heart.

The aorta 16 is a compliant arterial blood vessel that buffers and transfers pulsatile left ventricular output and contributes the largest component of total compliance of the arterial tree. The aorta 16 includes the ascending aorta 12, which begins at the opening of the aortic valve 7 in the left ventricle of the heart. The ascending aorta 12 and pulmonary trunk 11 twist around each other, causing the aorta 12 to start out posterior to the pulmonary trunk 11, but end by twisting to its right and anterior side. The transition from ascending aorta 12 to aortic arch 13 is at the pericardial reflection on the aorta 16. At the root of the ascending aorta 12, the lumen has three small pockets between the cusps of the aortic valve and the wall of the aorta, which are called the aortic sinuses, or the sinuses of Valsalva. The left aortic sinus contains the origin of the left coronary artery and the right aortic sinus likewise gives rise to the right coronary artery. Together, these two arteries supply the heart.

As mentioned above, the aorta 16 is coupled to the heart 1 via the aortic valve 7, which leads into the ascending aorta 12 and gives rise to the innominate artery 27, the left common carotid artery 28, and the left subclavian artery 26 emanating from the aortic arch 13 before continuing as the descending thoracic aorta 14 and further the abdominal aorta 15. References herein to the aorta may be understood to refer to the ascending aorta 12 (also referred to as the “ascending thoracic aorta,” or “aortic trunk”), aortic arch 13, descending or thoracic aorta 14 (also referred to as the “descending thoracic aorta”), abdominal aorta 15, or other arterial blood vessel or portion thereof.

Arteries, such as the aorta 16, may utilize blood vessel compliance (e.g., arterial compliance) to store and release energy through the stretching of blood vessel walls. The term “compliance” is used herein according to its broad and ordinary meaning, and may refer to the ability of an arterial blood vessel or prosthetic implant device to distend, expand, stretch, or otherwise deform in a manner as to increase in volume in response to increasing transmural pressure, and/or the tendency of a blood vessel (e.g., artery) or prosthetic implant device, or portion thereof, to recoil toward its original dimensions as transmural pressure decreases.

Arterial compliance facilitates perfusion of organs in the body with oxygenated blood from the heart. Generally, a healthy aorta and other major arteries in the body are at least partially elastic and compliant, such that they can act as a reservoir for blood, filling up with blood when the heart contracts during systole and continuing to generate pressure and push blood to the organs of the body during diastole. In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood may flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles and peripheral edema, for example, which may be caused by structural and/or functional cardiac abnormality. Such conditions can result in reduced cardiac output and/or elevated intra-cardiac pressures at rest or during stress.

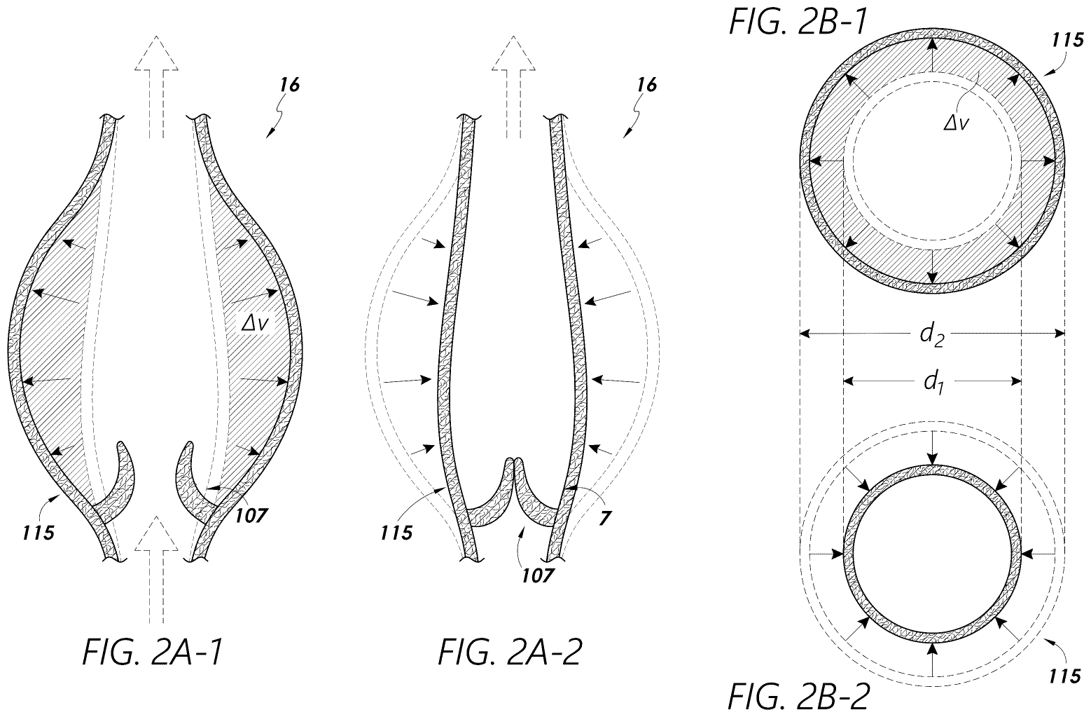

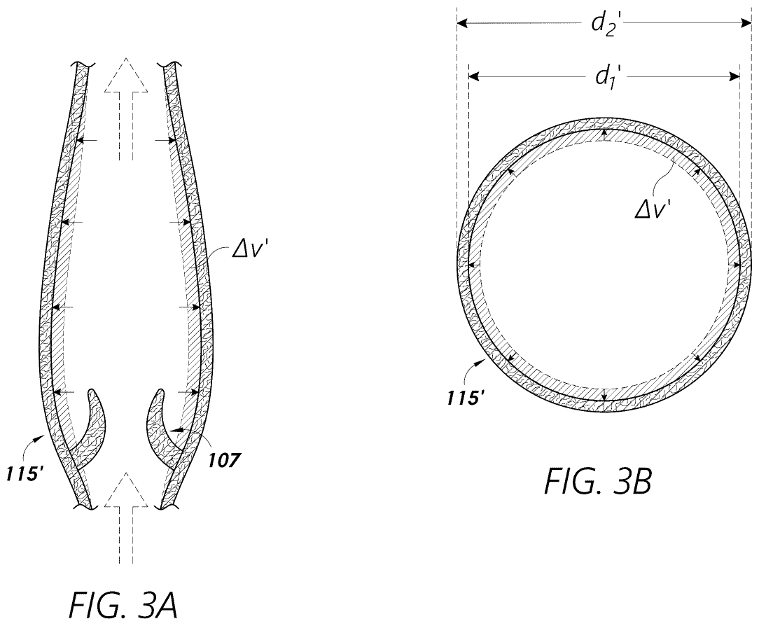

FIG. 1B-1 shows details of an example healthy aorta 16. FIGS. 2A-1 and 2B-1 show side and axial cross-sectional views, respectively, of the healthy aorta 16 of FIG. 1B-1 experiencing compliant expansion. FIGS. 2A-2 and 2B-2 show side and axial cross-sectional views, respectively, of the healthy blood vessel 16 of FIG. 1B-1 experiencing compliant contraction. FIG. 1B-2 shows details of an example stiff aorta 16′, whereas FIGS. 3A and 3B show side and axial cross-sectional views, respectively, of the stiff aorta 16′ of FIG. 1B-2 experiencing compromised expansion and/or contraction over a cardiac cycle.

The systolic phase of the cardiac cycle is associated with the pumping phase of the left ventricle, while the diastolic phase of the cardiac cycle is associated with the resting or filling phase of the left ventricle. As shown in FIGS. 2A-1 and 2B-1, with proper arterial compliance, an increase in volume Δv will generally occur in an artery when the pressure in the artery is increased from diastole to systole. With respect to the aorta, as blood is pumped into the aorta 115 through the aortic valve 107 (see FIG. 2A-1), the pressure in the aorta increases and the diameter of at least a portion of the aorta expands. A first portion of the blood entering the aorta 115 during systole may pass through the aorta during the systolic phase, while a second portion (e.g., approximately half of the total blood volume) may be stored in the expanded volume Δv caused by compliant stretching of the blood vessel 115, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance may have a significant effect on perfusion and/or blood pressure in some patients. For example, arteries with relatively higher compliance may be conditioned to more easily deform than lower-compliance arteries under the same pressure conditions. Compliance (C) may be calculated using the following equation, where Av is the change in volume (e.g., in mL) of the blood vessel, and Δp is the change in pulse pressure between systole to diastole (e.g., in mmHg):

$$C = \frac{Δ v}{Δ p} \qquad ( 1 )$$

Aortic stiffness and reduced compliance can lead to elevated systolic blood pressure, which can in turn lead to elevated intracardiac pressures, increased afterload, and/or other complications that can exacerbate heart failure. Aortic stiffness further can lead to reduced diastolic flow, which can lead to reduced coronary perfusion, decreased cardiac supply, and/or other complications that can likewise exacerbate heart failure.

Healthy arterial compliance may cause retraction/recoil of the blood vessel wall inward during diastole, thereby creating pressure in the blood vessel to cause blood to continue to be pushed through the artery 115 when the valve 107 is closed. For example, during systole, approximately 50% of the blood that enters the artery 115 through the valve 107 may be passed through the artery, whereas the remaining 50% may be stored in the artery, as enabled by expansion of the vessel wall. Some or all of the stored portion of blood in the artery 115 may be pushed through the artery by the contracting vessel wall during diastole. For patients experiencing arterial stiffness that causes lack of compliance, their arteries may not operate effectively in accordance with the expansion/contraction functionality shown in FIGS. 2A-1, 2B-1, 2A-2, and 2B-2.

As shown in FIG. 1B-2, the aorta 16′ tends to change in shape of the function of age, resulting in a higher degree of curvature and/or tortuosity over time. As the vasculature of a subject becomes less elastic, arterial blood pressure (e.g., left-ventricular afterload) becomes more pulsatile, which can have a deleterious effect, such as the thickening of the left ventricle muscle and/or diastolic heart failure. Stiffness in the aorta and/or other blood vessel(s) can occur due to an increase in collagen content and/or a corresponding decrease in elastin. While stiff/non-compliant blood vessels can generally suffer from a lack of elasticity in the walls thereof, as shown as causing compromised/reduced stretching and volume change Av′, such vessels can maintain some amount of flexibility/bendability, such that reshaping of the blood vessels can occur without necessarily requiring the stretching of the walls of the blood vessel.

Generally, the majority of aortic compliance is provided in the ascending aorta 12 with respect to healthy anatomy. Furthermore, calcification frequently occurs in the area of the ascending aorta 12, near the aortic arch 13 and the great vessels emanating therefrom. Such anatomical areas can experience relatively higher stresses due to the geometry, elasticity, and flow dynamics associated therewith. Therefore, implantation/deployment of compliance-enhancing, non-circular stent implant devices of the present disclosure can advantageously be in the ascending aorta 12 in some cases. While relatively less calcification tends to occur in the descending 14 and abdominal 15 aorta, implant devices of the present disclosure can advantageously be implanted/deployed in such areas as well for the purpose of smoothing arterial pressure/flow. Examples of the present disclosure provide compliance-enhancing, vessel-bending implant devices, which may be implanted in one or more locations in a compromised aorta and/or other vessel(s). For example, FIG. 1B-2 shows example positions of vessel-bending implant devices 101 including features disclosed herein implanted in various areas of an aorta 16′.

### Flow-Leveling Stent Implant Devices

Arterial compliance helps to level-out the otherwise strongly-pulsatile pressure and/or flow of blood cyclically output from the left ventricle by absorbing energy during high-pressure systole and returning energy to the circulation during low-pressure diastole. Flow-leveling stent implants of the present disclosure can advantageously reduce pulsatile energy of arterial blood flow by absorbing energy during systole, thereby reducing pressure peaks in the arterial circulation. Such implants can further be configured to reshape a blood vessel segment to a bent/crimped configuration as pressure decreases (e.g., during diastole), thereby pushing blood flow through the blood vessel and increasing the valleys in the pressure waveform and further smoothing the pressure waveform. The term “stent” is used herein in accordance with its broad and ordinary meaning and may refer to any device configured to be implanted in a lumen of a blood vessel, the device having a tubular form forming a lumen through which blood can flow.

The present disclosure relates to systems, devices, and methods for reducing pulsatile flow in the aorta or other target blood vessel. In some examples, such devices comprise stents biased in an axially-bent/crimped shape/configuration. Such devices may serve to reduce pressure peaks during systole and/or increase flow during diastole. Flow-leveling stent implant devices disclosed herein can improve compliance characteristics of a target blood vessel and/or otherwise improve circulation. To the extent that stent implant devices disclosed herein increase compliance in a target blood vessel by increasing diastolic/low-pressure flow, examples of the present disclosure can provide improved perfusion of the heart muscle and/or other organ(s) of the body. Examples of the present disclosure can include stents that, when implanted, are configured to reshape the target blood vessel segment to an axially-bent form/shape. In some implementations, when bent flow-leveling stents of the present disclosure are in a biased bent shape within a blood during low-pressure conditions, such stents may have a flow conduit/lumen volume that is relatively decreased compared to a straightened-cylinder configuration thereof, which serves to force blood through the blood vessel segment by pushing the blood through the vessel as the vessel volume reduces in connection with stent bending induced by cyclical drops in blood pressure.

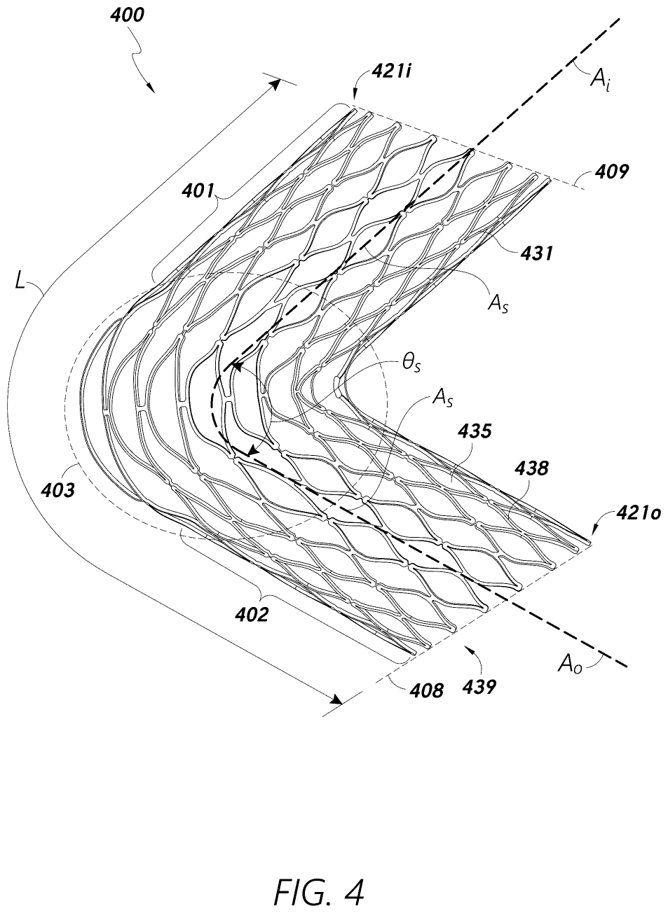

FIG. 4 shows a perspective view of a flow-leveling stent 400 in a relaxed, bent/crimped configuration in accordance with one or more examples. The stent 400 may be formed at least in part of a stent frame 431. The wall(s) of the frame 431 may be at least partially composed of struts 438 and/or stent openings/cells 435 between the struts 438. The tubular frame 431, which may form a wall around an axial channel 439, thereby defining the channel 439. The stent 400 may be an elongate/elongated stent, in that a length L of the stent is greater than a maximum diameter of the stent. As described herein, the frame wall(s) 431 of the stent 400 can be considered a single, circumferentially-wrapped wall, or may be considered to comprise multiple walls, or wall segments.

The stent 400 may include one or more relatively straight portions 401, 402, as well as one or more bends 403. As described herein, where stents or portions thereof are described as being straight, bent, and/or crimped, such description may be understood relative to an axis Aₛ of the stent, and/or with respect to a side view of the stent. That is, a stent segment described as straight may have a central axis that is substantially straight, whereas in a bent/crimped stent portion, the axis Aₛ of the stent may deflect or bend at an angle θ5, as shown in FIG. 4.

The stent 400 is shown with a single bend 403. However, it should be understood that flow-leveling stents of the present disclosure may have multiple bends, wherein each of the multiple bends may be generally in the same direction, or may be in opposite directions (e.g., a bent stent having a ‘z,’ or zigzag, shape). Furthermore, although a single bend 403 is illustrated as producing the axial deflection θₛ between the direction/dimension of the axis Aᵢ at an inflow end 421i of the stent 400 relative to the direction/dimension of the axis Aₒ at the outflow end 421o of the stent 400, in some implementations, the axial deflection θₛ is produced/achieved gradually over the length L of the stent, such as over a substantially continuous curve that extends a length of the stent 400 (e.g., forming a ‘c’/crescent-shaped stent, as opposed to a ‘v’-shaped stent).

The bend 403 may be positioned at the lengthwise center/midsection, as shown, such that the bent 403 represents a medial portion of the stent between the first 401 and second 402 straighter segments. Regardless of whether the bend angle θₛ of the stent 400 is provided by a discrete bend in a medial portion, or over a more gradual bend along the length of the stent, the degree to which the stent 400 provides flow-leveling and/or compliance-enhancing functionality may be based at least in part on the tightness/degree of the angle θₛ. References herein to bend angle of a stent may refer to the absolute angle measurement between the axes Aᵢ, Aₒ (e.g., approximately 75° in the example illustration of FIG. 4), or the absolute angle taken from 180° (e.g., approximately 105° in the illustrated example).

The inlet 421i and outlet 421o ends of the stent frame 431 may lie in parallel or non-parallel planes. For example, the planes 408, 409 of the ends of the stent frame 431 may be angled relative to one another by 180° minus the bend angle θₛ, wherein the planes 408, 409 are normal/orthogonal to the axes Aₒ, Aᵢ at the respective ends. Alternatively, the frame 431 may be designed such that the planes 408, 409 of the ends 421o, 421i, respectively, may be parallel and generally orthogonal to a line connected directly between an axial center of the frame at the inlet 421i and outlet 421o ends, respectively. Alternatively, as illustrated, the planes 408, 409 may be angled relative to one another by an angle between 0° and 180°−θₛ (i.e., 180° minus θₛ).

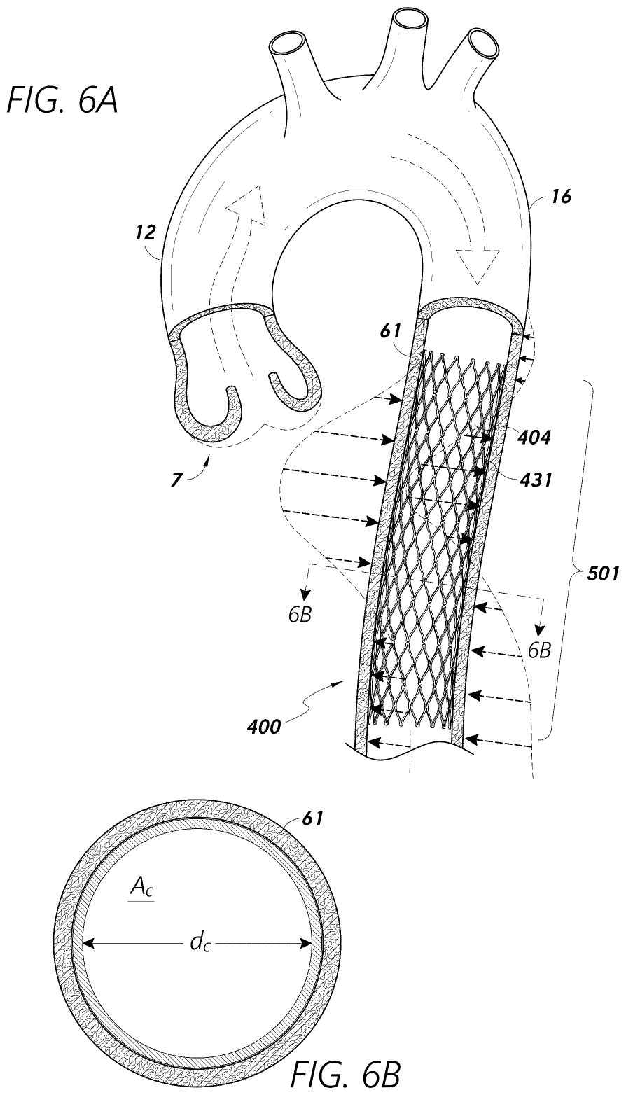

The shape/configuration of the stent 400 shown in FIG. 4 may represent a relaxed/resting configuration of the stent. That is, the stent frame 431 may have a shape-memory that biases the shape of the stent 400 to the bent/crimped configuration shown. Therefore, when the stent 400 is manipulated in some manner as to reshape the stent to a different shape/configuration, once such deforming forces are removed, the stent 400 may be inclined to return of its own accord to the bent configuration shown in FIG. 4. For example, where forces act upon the stent frame 431 in a manner as to deform/reshape the stent 400 to a more-straight cylindrical shape (see FIG. 6A), the frame 431 (e.g., struts thereof) may store energy that, when released as deforming/reshaping forces are removed, cause the stent frame 431 to return to the bent/crimped state shown. In some implementations, the angular deflection θₛ of the stent 400 may be an acute angle (less than 90°). In some implementations, the angular deflection θₛ may be approximately 90°, or some angular deflection between 90°-130°.

The frame 431 and/or wall(s) thereof may comprise an open-cell structure adapted to be expanded to secure the stent 400 to a blood vessel internal (or external) wall, such as through a pressure-fit deployment. Anchoring of the stent 400 may further be facilitated by one or more tissue anchors/barbs, and/or endothelialization of the frame 431 to the vessel tissue over time. The stent 400 may be elastically deformable between the relaxed bent/crimped configuration shown in FIG. 4 and a more-straight configuration (see FIG. 6A), with the stent 400 biased toward the bent configuration. In some examples, the stent frame 431 may comprise a shape-memory material, such as nitinol, which provides shape-memory biasing of the frame 431 towards the bent configuration.

As the stents of the present disclosure produce flow-leveling within a blood vessel by manipulating/reshaping the native blood vessel walls, compliance can be increased in the target blood vessel without requiring blood vessel grafting or resection. Therefore, compared to blood flow solutions involving blood vessel grafting/resection, examples of the present disclosure can provide a solution that avoids the risks that may be associated with cutting of the vessel and/or devices grafted in/to such vessels, which may present risk of rupture and blood leakage outside of the circulatory system. Hazards associated with extravascular arterial blood leakage, such as within the abdominal and/or chest cavity, can include the risk of serious injury or death.

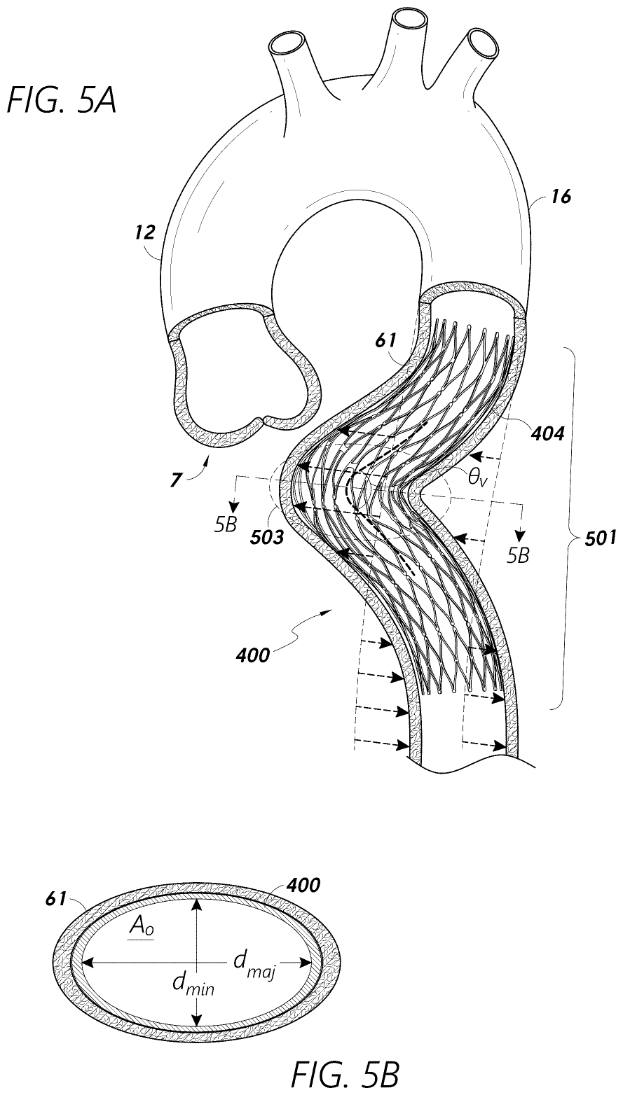

FIGS. 5A and 5B show side cutaway and axial cross-sectional views, respectively, of the flow-leveling stent 400 of FIG. 4 implanted in a segment of an aorta 16 of a patient, the flow-leveling stent 400 being configured in a crimped/bent configuration within the blood vessel 16 in accordance with one or more examples. For example, the stent 400 may be implanted in the blood vessel 16 (e.g., descending aorta), wherein the shape memory of the bent/crimped stent 400 causes the blood vessel segment 501 in which the stent 400 is implanted to be reshaped in a manner as to conform to some degree to the shape memory configuration/shape of the stent 400. Due to aging or other causes, the aorta 16 shown may be a relatively stiff aorta, wherein the aorta forms a tubular vessel with a relatively fixed/constrained wall area. The device 400 is configured to improve compliance of such a stiff vessel/segment.

The stent 400 may be configured to be percutaneously delivered to a blood vessel in a compressed delivery configuration. Once within the blood vessel lumen at the target deployment site, the stent 400 and/or frame 431 thereof may be configured to be radially expanded into direct surface contact with the blood vessel wall (e.g., the inner wall of an aorta segment). In some examples, the stent 400 may be configured to be expanded such that the perimeter of the stent 400 approximates and/or exceeds a perimeter of the blood vessel portion where the stent 400 is implanted, at least immediately prior to deployment/expansion of the stent. In some cases, a stent configured to expand to a greater perimeter than the native blood vessel may provide improved traction and/or resistance to migration within the blood vessel. The bent configuration/biasing of the stent 400 may further serve to secure the stent 400 in-place within the target blood vessel by impeding axial migration through friction and other wall contact forces. The stent wall and/or a portion of the stent wall may be configured to be endothelialized to the blood vessel wall.

The dimensions and/or shape of the stent 400 may vary based on the particular application and/or target implantation anatomy. For example, the stent length L may be selected to extend over all or a portion of an identified non-compliant length of a target blood vessel. As an example, for a stent configured for deployment in an aorta, the length L may be between 1-15 cm. However, other sizes and/or shapes are also within the scope of this disclosure. The stent major axis dₘₐⱼ and minor axis dₘᵢₙ, when averaged, may be approximately equal to the diameter of the native blood vessel, or slightly greater than the native blood vessel diameter prior to stent implantation.

As shown in FIG. 5A, the blood vessel segment 501 may be bent/crimped by the stent 400 in a manner as to form one or more bends 503, as shown. FIGS. 5A and 5B (and 6A and 6B) can be understood with reference to the image of FIG. 4 and related description above. In some instances, the bend(s) 503 may produce an axial deflection θₛ that may be an angle that is at least slightly greater than the relaxed, non-implanted axial deflection θₛ of the stent 400. That is, the resulting axial deflection θₛ of the blood vessel segment 501 may be less than the relaxed biased deflection θₛ of the stent 400 due to the resistance of the blood vessel segment 501 to reshaping, which may impede/prevent the stent 400 from reshaping the blood vessel segment 501 to exactly match the pre-shaped angular deflection θₛ of the stent 400. The spring-biased bend 403 can act as a resistor to flow and pressure forces of high-pressure circulation, thereby consuming energy from such circulation and leveling-out the pressure and/or flow amplitudes thereof.

The crimped/bent configuration of the stent 400 and blood vessel segment 501 in the image of FIG. 5A may be associated with a relatively-low-pressure state of the circulation within the blood vessel 16, wherein the pressure levels within the blood vessel segment 501 are not so great as to prevent the stent 400 from reshaping the blood vessel segment 501 in the manner illustrated. For example, FIGS. 5A and 5B may be associated with the diastolic phase of the cardiac cycle, in which the luminal pressure levels in the aorta 16 are relatively low.

FIG. 5B shows an example cross-sectional shape/form of the stent 400 in the relaxed bent/crimped state thereof at one or more positions along the length of the stent 400, such as at or near the bend 403 of the stent 400. Although a particular non-circular/oval cross-sectional shape is shown in FIG. 5B with respect to the stent 400, it should be understood that in some implementations, in the biased/bent configuration of the stent 400, the axial cross-sectional shape thereof is substantially circular in one or more lengthwise portions/areas thereof. In some implementations, as shown in FIG. 5B, the bent/crimped configuration of the stent 400 may be associated with a non-circular axial cross-section thereof in one or more areas of the stent. Where the stent 400 forms a non-circular (e.g., oval) cross-sectional shape, such shaping of the stent can force a corresponding non-circular cross-sectional shape in the blood vessel wall(s) 61 surrounding the stent frame, as shown.

FIGS. 6A and 6B show side cutaway and axial cross-sectional views, respectively, of the flow-leveling stent 400 of FIG. 5A implanted in the blood vessel segment 501, the flow-leveling stent 400 being configured in a straightened configuration in accordance with one or more examples. The configuration of the stent 400 in the straightened configuration shown in FIG. 6A may correspond to a high-luminal-pressure state of the cardiac anatomy. For example, the image of FIG. 6A may be associated with the systolic phase of the cardiac cycle, wherein the aortic valve 7 is open to allow high-pressure, pulsatile flow from the left ventricle (not shown) into the aorta 16.

As described above, the stent 400 may have a bent/crimped biased shape, such that in the absence of reshaping forces on the stent, the stent 400 may be inclined to reshape the blood vessel segment 501 to a bent configuration as shown in FIG. 5A. However, in high-pressure conditions within the blood vessel 16, such luminal pressure may exert forces on the blood vessel walls 61 and/or stent frame 431 (e.g., on a covering of the stent that is coupled to the frame 431) that force the shape thereof to a more-straight cylindrical shape as shown in FIG. 6A. For example, the luminal pressure forces may press against the inner diameter/wall(s) 61 of the blood vessel segment 501 to cause a straightening thereof, wherein such straightening of the blood vessel 501 may in-turn apply force and/or push against the stent frame 431 in a manner as to overcome the bias of the stent 400 in the bent shape and force the stent 400 to assume a more-straight cylindrical shape. In addition, or alternatively, the high luminal pressure forces in the flow conduit of the stent 400 may press against the frame 431 and/or other component(s) (e.g., covering) of the stent 400 to force the straightening thereof, wherein such straightening of the stent 400 may occur in parallel/correlation with the straightening of the blood vessel segment 501 and/or may force the blood vessel segment 501 into the straightened configuration. For example, in some implantations, the stent 400 may include a covering on inner and/or outer diameter surface(s) of the stent frame 431, wherein the covering provides surface area for luminal pressure forces to press against, wherein such forces against the covering, due to the coupling of the covering with the stent frame 431, cause the reshaping of the stent frame 431. Although FIG. 6A shows a substantially straight configuration of the stent 400 and blood vessel segment 501, it should be understood that the configuration of the stent and blood vessel segment in the more-straight, high-pressure configuration may not be fully straight, but rather may be a shape that is straighter than the shapes shown in FIG. 5A but less straight than the shapes shown in FIG. 6A.

The reshaping of the stent 400 from the bent configuration to the relatively-straight configuration can be through the action of fluid pressures on the blood vessel wall through open cells of the stent. Alternatively or additionally, reshaping forces may be applied to the stent frame 431 through application on a fluid-tight covering coupled to the frame 431. After the high-pressure/flow conditions of systole force the blood vessel segment 501 and stent 400 to the relatively-straight configuration of FIG. 6A, the advent of cyclical low-pressure/flow conditions can initiate a return of the blood vessel segment 501 and stent 400 to the bent shape/configuration by allowing for the release of mechanical spring energy from the flexible stent frame 431.

The cyclical bending of the blood vessel segment 501 in diastole as in FIG. 5A, straightening of the blood vessel segment 501 in systole as in FIG. 6A, and subsequent re-bending of the blood vessel segment 501 as the cardiac cycle transitions back to diastole, can provide a desired flow-leveling effect. For example, as high-pressure blood flow enters the blood vessel segment 501 during systole, the flow may apply pressure forces against the bent walls 404 of the stent 400 and/or blood vessel segment 501, causing deflection of such bent wall(s) towards a straighter configuration. Furthermore, the increased luminal pressure may provide radially-outward forces against the blood vessel walls 61 and/or stent 400 in other areas thereof that may further cause straightening of the segment 501 and stent 400. That is, for relatively-high pressure and flow through the blood vessel segment 501, the forces associated therewith may push/manipulate the blood vessel walls 61 and/or stent frame 431 to assume a shape of minimal/lesser resistance to such flow. Such shape of minimal/lesser resistance may generally be a straighter cylindrical shape compared to the bent shape shown in FIG. 5A. The process of straightening the blood vessel segment 501 and/or stent 400 may absorb energy associated with the overcoming of the biased shape-memory of the stent 400 to transition the stent 400 from the bent shape of FIG. 5A to the straighter shape of FIG. 6A. The work necessary to transition the stent 400 and/or blood vessel segment 501 to the straighter shape may be performed by the flow rate and pressure forces of the circulation. Therefore, by forcing the blood vessel segment 501 into the bent shape, such that the systolic blood flow must navigate a more tortuous path, the resistance to flow provided by the tortuous shape of the stent 400 and/or blood vessel segment 501 causes a reduction in flow rate and/or pressure of the blood circulation, thereby reducing the pulsatility of the blood flow, which can provide certain physiological benefits as described in detail above.

The spring-biasing of the stent 400 towards the bent configuration can allow for the stent 400 to store mechanical energy in the form of spring forces when the stent is deformed from the bent configuration towards the more-straight configuration. The flexible stent 400 can be placed within a curved portion of the aorta 16, either within the ascending aorta 12 or the descending aorta 14. The flexibility of the stent 400 allows it to assume and/or conform to the bent shape of the aortic portion in which it is placed. However, during systole, as shown in FIG. 6A, the expansion forces applied to the stent 400 facilitate axial straightening of the same aortic portion to some degree, wherein the stent 400 reverts back to its bent free state in diastole, as shown in FIG. 5A. Thus, each pulse of the cardiac cycle is associated with a cycle of straightening and bending of the aortic portion in which the stent 400 is implanted, resulting in energy loss that improves aortic compliance, effectively producing a reduction in stiffening of the same aortic portion. The degree of stent flexibility can be selected/determined to be able to apply higher flexibility (e.g., reduced stability) of a bent/curved portion of an aged, tortuous aorta.

As mentioned, the transition between the bent configuration and the straighter configuration reduces peaks in the pressure and/or flow waveforms of the blood circulation in the blood vessel 16. Furthermore, the transition back from the straighter configuration shown in FIG. 6A to the bent configuration of FIG. 5A can further level-out the flow through the blood vessel 16 and/or segment 501 thereof. For example, the transition of the blood vessel segment 501 from the straighter configuration to the bent/crimped configuration can push blood through the blood vessel segment 501 and/or out of the output end 421o of the flow channel 439 of the stent 400. Such pushing of the blood through the flow channel 439 can be caused at least in part by one or more sidewalls of the stent 400 and/or blood vessel segment 501 deflecting into the previously-straight flow channel through the segment 501 and stent 400. Such deflection can push against blood disposed in the flow channel, thereby forcing such blood through the segment 501. For example, blood disposed within the segment 501 downstream of the bend 503 may be pushed out the outflow end 421o of the stent. Such pushing of blood through the segment 501 may increase blood pressure and flow in one or more areas of the blood vessel 16. Therefore, the low points of the pressure and/or flow waveforms may be brought-up to some degree at least with respect to one or more areas of the blood vessel 16, such that the waveforms may be further leveled in a manner as to reduce the pulsatility of the of the circulation in the blood vessel.

The leveling of blood flow in the blood vessel 16 may further be facilitated by a change in volume of the blood vessel segment 501 between the bent configuration of FIG. 5A and the straighter configuration of FIG. 6A. That is, the flow channel 439 of the stent 400 and through the blood vessel segment 501 in the bent configuration of FIG. 5A may have a volume that is less than the volume of the same length of flow channel when the segment 501 is in the straighter configuration of FIG. 6A. For example, the cross-sectional area of the stent 400, and therefore the blood vessel walls 61 in the blood vessel segment 501, may be non-circular in one or more areas thereof when the stent 400 is in the bent configuration, as with a kink in a hose. An example non-circular cross-sectional shape of the stent 400 and blood vessel 61 is shown in FIG. 5B, corresponding to a portion of the stent 400 and blood vessel 61 in the bent configuration. As the stent 400 transitions to the straighter configuration shown in FIG. 6A, the cross-sectional area thereof may become more circular, as reflected in FIG. 6B. The change in cross-sectional area of the stent 400, which results in a commensurate change in cross-sectional area of the blood vessel walls 61 around the stent when the size of the stent 400 is sufficiently matched to the size of the blood vessel, can result in a dampening effect on the pressure and/or flow through the blood vessel segment 501 during systole and/or increase of pressure and/or flow through the blood vessel segment 501 during diastole. For example, in transitioning between non-circular (e.g., oval-shaped, as illustrated) and more-circular cross-sectional shapes, the stent frame 431 can absorb energy during systole and return such energy to the blood circulation during diastole, the mechanics of which are described in greater detail below.

By implementing the change in cross-sectional area of the flow channel 439 in connection with cardiac cycling, as reflected in FIGS. 5B and 6B, the stent 400 can cause a change in cross-sectional area/volume of the blood vessel segment 501 in one or more areas thereof without requiring stretching/compliance of the blood vessel wall 61, which can promote more-even flow of blood through the blood vessel throughout the cardiac cycle. By way of demonstration, with respect to a blood vessel having a relatively fixed perimeter, wherein the blood vessel wall does not expand sufficiently due to stiffness and/or other factors of non-compliance, generally, the greatest area/volume of the blood vessel may be present/achieved when the blood vessel wall forms a circular cross-sectional shape, which may maximize the cross-sectional area and volume of the blood vessel. The cross-section of FIG. 6B corresponds to a circular/more-circular cross-sectional shape, such that the area A_{c} thereof is greater for the given perimeter/wall-length of the blood vessel wall 61.

Diverging from a circular cross-sectional shape can produce a cross-sectional area/volume for a blood vessel that is less than the more-circular area A_{c} shown in FIG. 6B. The non-circular shape of the stent 400 in FIG. 5B may have a shape that resembles an oval/ellipse (or prolate, oblate, elongate, and/or pointed ellipse or other closed curve), which produces the cross-sectional area A_{c} that is less than the area A_{c} with the same blood vessel wall/perimeter length 61. The oval shape of the stent 400 in FIG. 5B may have a major-axis dimension dₘₐⱼ that is greater than a minor-axis dimension dₘᵢₙ thereof. Due to the area Aₒ of the oval stent/vessel being less than the area A_{c} of the more-circular configuration, transitioning from the more-circular shape to the less-circular shape, can provide a reduction in area/volume of the blood vessel, and therefore provide compliance characteristics without the need for elasticity in the blood vessel wall tissue. Such manipulation of the blood vessel shape can introduce volumetric change in the blood vessel in response to the typical changes in pressure experienced during the cardiac cycle, thereby increasing cardiac efficiency and reducing pulsatile load.

In some implementations, the stent 400 may be considered an oval stent as having a non-circular axial cross-section in one or more segments thereof when in the relaxed, biased configuration of the shape-memory-biased frame 431. The term “oval” is used herein according to its broad and ordinary meaning and may be used substantially interchangeably with the term “ellipse” and/or “oblong,” which terms are likewise used according to their broad and ordinary meanings. The term “oval” may be used to refer to any non-circular closed curve having major and minor axes, the major axis being greater than the minor axis. With respect to “oval”-shaped stents disclosed herein, such stents may have relatively flatter minor-axis sidewalls (compared to curved major-axis end walls), wherein the sidewalls may bow radially outward, and/or may be deflected/curved radially inward so as to produce external concavity and internal convexity in such sidewalls (e.g., forming a peanut-shaped stent).

The stent 400 may be delivered to the target implantation site in a radially-compressed delivery configuration within a delivery catheter/sheath. Once deployed from the delivery catheter/sheath, the stent 400 may be configured to self-expand according to the shape memory thereof being configured to self-expand to the radially-expanded configurations shown in FIGS. 4, 5A, and/or 5B. Additionally or alternatively, the stent 400 may be radially expanded using a balloon device inflated within the flow channel 439 of the stent 400.

### Flow-Leveling Clamp Devices

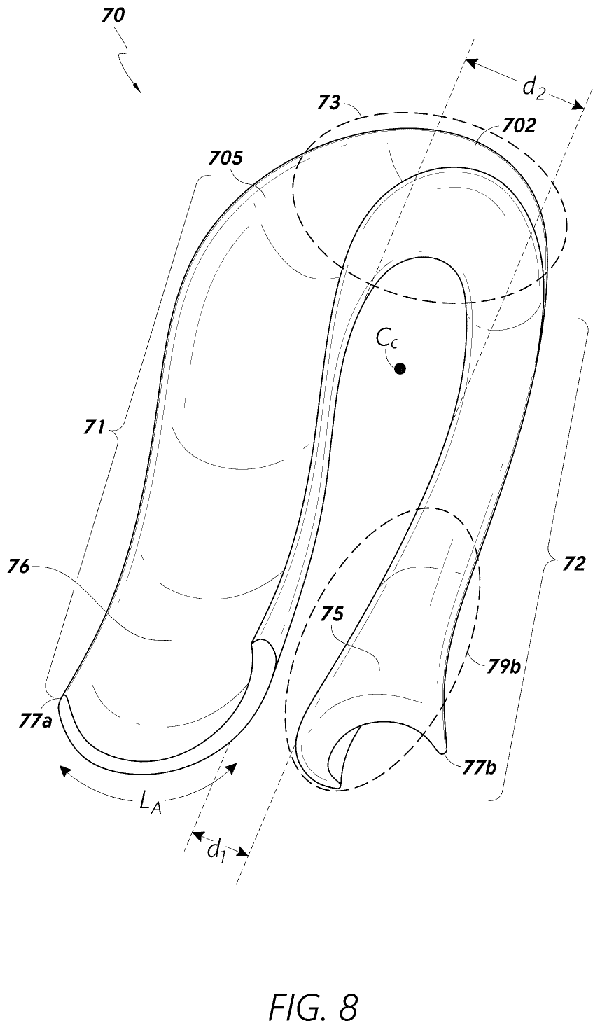

In some implementations, example flow-leveling implant devices of the present disclosure comprise flexible devices configured to be implanted/deployed along the curvature between the ascending and descending portions of an aorta. Such implant devices can comprise clamp-type devices. FIGS. 7-1 and 7-2 show a clamp-type flow-leveling implant device 70 implanted in an aortic arch 13, the clamp device 70 shown in crimped and expanded configurations, respectively, in accordance with one or more examples. FIG. 8 shows a perspective view of the clamp implant device 70 in accordance with one or more examples.

As described above, examples of the present disclosure provide spring-biased implant devices configured to remodel a blood vessel segment into a relatively bent/crimped state in low-pressure conditions, wherein increases in luminal pressure in the blood vessel segment cause the blood vessel to overcome the biasing of the implant device to transition the blood vessel segment to a less-bent/crimped configuration. Transition between the bent and less-bent configurations of the blood vessel serves to level-out the pulsatility of blood flow and/or increase flow/compliance in the blood vessel. While some examples are illustrated as achieving blood vessel bending/crimping by the use of axially-bent stents, it should be understood that blood vessel bending/crimping can be implemented using other types of implant devices. Furthermore, although some examples are presented herein in the context of stents or other implant devices implanted in the descending thoracic or abdominal aortas, or other relatively straight blood vessel segments, it should be understood that bending/crimping implant devices of the present disclosure may be configured to effect such bending/crimping in curved blood vessel segments, such as in the aortic arch. The flow-leveling implant device 70 shown in FIGS. 7-1 and 7-2 represents an example of a flow-leveling, clamp-type implant device configured to bend/crimp a target blood vessel, wherein the device 70 is configured to be deployed in, and cause crimping/bending of, the aortic arch or other curved/tortuous blood vessel anatomy.

The implant device 70 may have an elongated form configured to span at least a portion of the aortic arch 13 of the patient. For example, as shown, the implant device 70 may be disposed on/over an interior of the aorta 16 along an inner radial wall 701 along the arch 13. For example, the implant 70 may include a first (e.g., distal) relatively straight/long arm/portion 71, which may be considered an ascending aorta portion of the implant and may be configured to be disposed at least partially within the aortic trunk 12 of the ascending aorta. The implant 70 may further include a second (e.g., proximal) relatively straight/long portion 72, which may be considered a descending portion of the implant 70 and may be configured to be disposed was partially within the descending aorta 14, such as the descending thoracic aorta, as shown. The ascending arm/portion 71 and descending arm/portion 72 may generally be oriented in the same direction, such as within 45°, 30°, and/or 15° of parallel alignment when the implant 70 is in a relaxed, biased configuration/shape as shown in FIG. 8.

The ascending 71 and descending 72 arms/portions of the implant 70 may be coupled by a bend/curve portion 73 having a vertex or apex 702 where the inflection of the bend 73 transitions between angling towards the distal arm 71 and angling towards the proximal arm 72. The bend 73 can be semicircular in shape, as shown, wherein the curved shape of the bend 73 has a radius of curvature rₐ with respect to a center C_{c} of the curve (e.g., center of curvature of the bend 73). In the relaxed, biased configuration, the implant 70 may have a shape with respect to a side view thereof that resembles that of a horseshoe, ‘u,’ wishbone, or similar shape. The clamp device 70 may be considered to have a horseshoe shape where, in the relaxed biased configuration, the tissue-contact surfaces/portions 75a, 75b of the distal 71 and proximal 72 arms, respectively, are separated by a separation distance/gap d₁ that is less than a separation distance/gap d₂ in an area closer to the bend 73; that is, the distance between the first arm 71 and the second arm 72 may increase moving from the primary tissue-contact areas 79 towards the bend region/area 73.

The bend/curve 73 of the implant 70 may be spring-biased to position the ends 77a, 77b of the respective ascending 71 and descending 72 arms/portions to a relatively close separation distance d₁, wherein the application of pulling forces to pull the ends 77 away from one another to increase the separation distance d₁ is resisted by the spring-biased shape-memory of the implant 70 and causes energy to be stored in the implant 70, such as in the spring-biased bent/curved portion 73. That is, where the ends 77 and/or elongated arms 71, 72 are forced apart, the bent/curve portion 73 may exert forces on the elongated arms 71, 72 to return the ends 77 to the biased distance d₁.

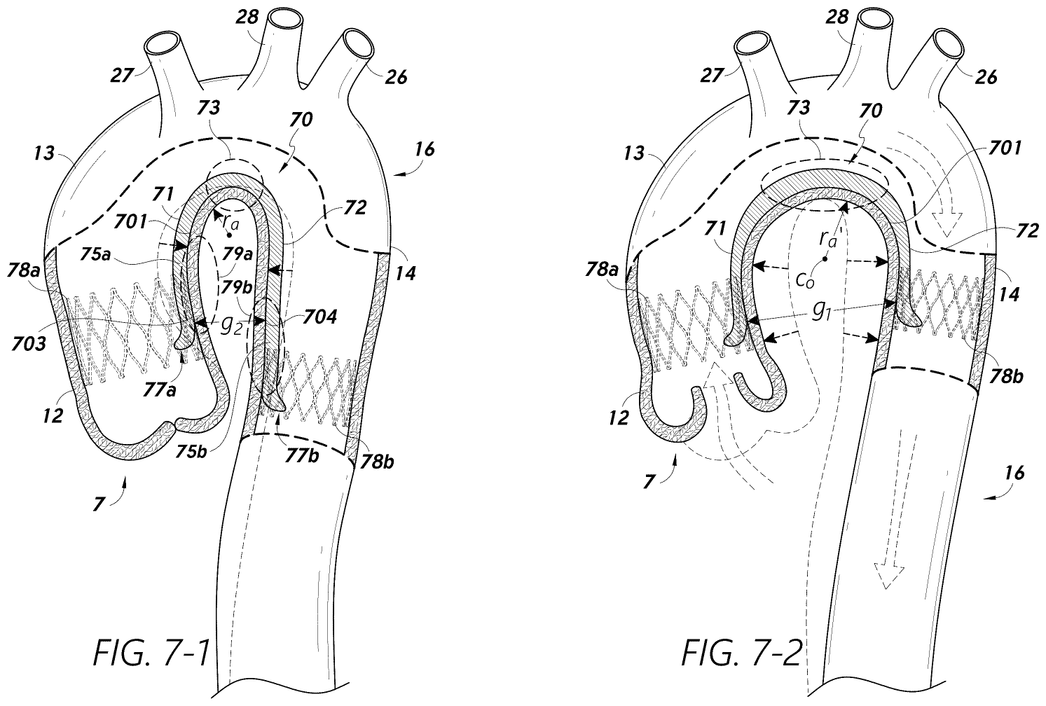

The biasing of the implant device 70 in the folded/crimped configuration shown in FIG. 8, when the implant 70 is implanted over an aortic arch as shown in FIG. 7-1, can cause the aortic arch (or other target vasculature) to be crimped in a manner as to reduce a radius of curvature rₐ thereof and draw ascending 703 and descending 704 portions of the inner radius of the aorta relatively closer together to a separation distance g₂, as shown in FIG. 7-1. That is, the spring-biasing of the bend/curve portion 73 of the implant 70 may apply force against the inner wall 701 of the aorta, such as at the contacts thereof with the end portions 77a, 77b of the implant 70, to cause the crimping/folding of the vascular anatomy.

The arms of the device can comprise flexible portions that, when implanted, extend along or toward the ascending aorta 12 and the descending aorta 14, respectively, as illustrated. During systole, the implant 70 may be deformed such that the arms 71, 72 become spaced relatively further from each other to the expanded gap distance g₂′, wherein in diastole the arms return/revert back toward each other to the shortened gap distance g₂. Thus, during each cardiac cycle, the aortic arch 13 may follow the open and closed states of the device 70 to improve flow therethrough. In some implementations, the device 70 can be activated by the pressure changes in the aorta 16 during the cardiac cycle. In alternative examples, the device 70 can be activated in synchronization with measured electrical signals (e.g., ECG) of the heart. Advantageously, the various solutions for crimping the aortic arch using an implant device such as the clamp 70 can take advantage of the aorta's flexibility in the axial direction to change shape between the systolic and diastolic phases of each cycle, so as to further improve blood flow therethrough.

The first/distal 71 and second/proximal 72 arms can have interior tissue-contact surface 75. The tissue-contact surfaces 75a, 75b are opposite-facing and face one another. The bend portion 73 can couple the first arm 71 to the second arm 72, the bend portion 73 being spring-biased to the crimped configuration shown in FIG. 8, in which the tissue-contact surface 75a of the first arm 71 and the tissue-contact surface 75b of the second arm 72 are a first distance da apart. Application of force to pull the first arm 71 and the second arm 72 apart such that the first tissue-contact surface 75a and the second tissue-contact surface 75b are a second distance apart that is greater than the first distance d₁ causes the bend portion 73 to exert force on at least one of the first arm 71 or the second arm 72 to urge the first arm 71 and the second arm 72 closer together.

FIG. 7-1 shows the implant 70 implanted in the aorta 16 in a relatively low-pressure phase (e.g., diastole), whereas FIGS. 7-2 shows the implanted device 70 during a relatively-high pressure phase (e.g., systole), which may correspond with the pumping of the left ventricle and opening of the aortic valve 7. The increased luminal pressure in the high-pressure phase of FIG. 7-2 may cause the aortic arch 13 and/or associated anatomy to reshape towards a more natural shape and/or radius rₐ′, wherein such reshaping of the aorta 16 overcomes the biasing of the bend/curve 73 of the implant 70 to accommodate the expanded radius of curvature rₐ′.

In the crimped configuration shown in FIG. 7-1, the aortic arch 73 may have a total volume that is reduced by some amount relative to the uncrimped configuration shown in FIG. 7-2, irrespective of radially-compliant stretching of the aortic arch blood vessel tissue. That is, the tubular shape of the arch 13 in the crimped state may have a reduced volume relative to the non-crimped natural tubular shape of the arch in the absence of the flow-leveling implant 70. Furthermore, the crimped configuration of the vessel 16 may present a more tortuous flow path for blood flow therein relative to the un-crimped vessel that has a greater and more constant radius of curvature around the arch 13.

Due to the change in volume and/or tortuosity between the crimped configuration of FIG. 7-1 and the un-crimped configuration of FIG. 7-2, cyclical transition between such configurations, as caused by the spring biasing of the implant device 70, can affect the blood circulation through such blood vessel segments in a manner as to provide a leveling effect with respect to pressure and/or flow waveforms of the circulation. For example, at the advent of systole, the luminal flow may encounter a relatively tortuous flow path through the crimped arch 13, and may further enter a blood vessel lumen that has a relatively reduced volume due to the crimping thereof. The pressure and flow of the systolic circulation may overcome the biasing of the implant device 70 to un-crimp the device as in FIG. 7-2. Such overcoming of the bias of the device 70 may be achieved by the pulling of the vessel walls on the arms 71, 72, such vessel walls being acted upon by the pressure and/or flow forces of the circulation. The un-crimping of the device 70 as in FIG. 7-2 may cause energy to be absorbed in the device 70 and/or may reduce peak pressure and/or flow parameters of the aortic circulation in a manner as to provide a leveling effect as described in detail herein.

As the pressure in the vascular lumen subsides as the cardiac cycle transitions from systole to diastole, the forces within the blood vessel lumen may be insufficient to overcome the biasing of the implant device 70, and therefore the implant 70 may be permitted to re-crimp the aortic arch 13, thereby altering/reducing the volume thereof and/or changing the geometry of the arch 13 in a manner as to push blood through the arch 13 and down the descending aorta 14. For example, crimping of the arch 13 can deflect the blood vessel walls thereof to push at least a portion of the blood disposed in the descending portion 14 of the arch downstream. Furthermore, reduction in luminal volume may force the blood downstream due to the aortic valve 7 being closed. Therefore, the crimping action of the implant 70 may increase diastolic flow downstream of the implant 70, thereby further leveling the pressure and/or flow waveforms of the circulation in an advantageous manner.

The ‘u’/horseshoe shape of the implant 70 may serve to anchor/hold the implant 70 in place in the aortic arch area of the aorta 16. In some examples, the implant 70 may include certain anchoring features 78, such as one or more stent-type anchors, barbs, sutures, or other anchoring means to further secure the implant 70 in-place in the aorta 16. For example, FIGS. 7-1 and 7-2 show optional distal 78a and proximal 78b anchors (e.g., stents), which may be associated with distal 77a and proximal 77b ends of the implant 70. The anchors 78 may be mechanically coupled to the device 70, or may be an integral form therewith. For example, the horseshoe/‘u’-shaped clamp portion 705 (see FIG. 8) of the implant 70 and the stent-type anchors 78 may be cut from a common tube (e.g., metal, plastic), such that the anchors 78 and the clamp form 705 are of a unitary construction.

In some examples, the clamp portion 705 of the implant 70 has a radially-outward concavity (radially-inward convexity), such that the form thereof conforms laterally La to the radius of the blood vessel, as shown in FIG. 8. Such concavity may span the entire length of the clamp form 705, or one or more portions thereof. The concavity of the implant 70 may provide a spoon-like form, which may advantageously reduce impingement or damage on/to the blood vessel tissue. The laterally-concave shape of the implant 70 may further facilitate anchoring thereof in the desired position in the aortic arch by allowing for relatively improved conformality with the shape of the target vascular anatomy. Although the device 70 is illustrated as an elongated strip/structure formed into distal and proximal arms connected by a bend, other designs may be implemented that may include any structure, such as a stent or the like, that is configured to reshape a blood vessel arch/curve to a crimped configuration.

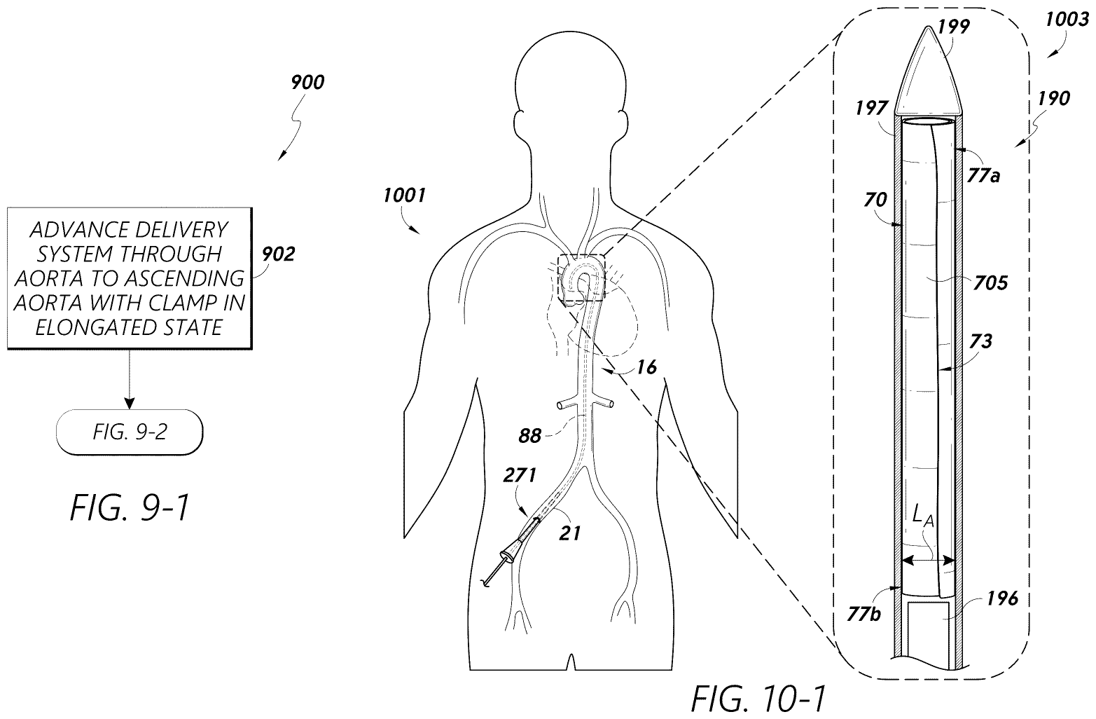

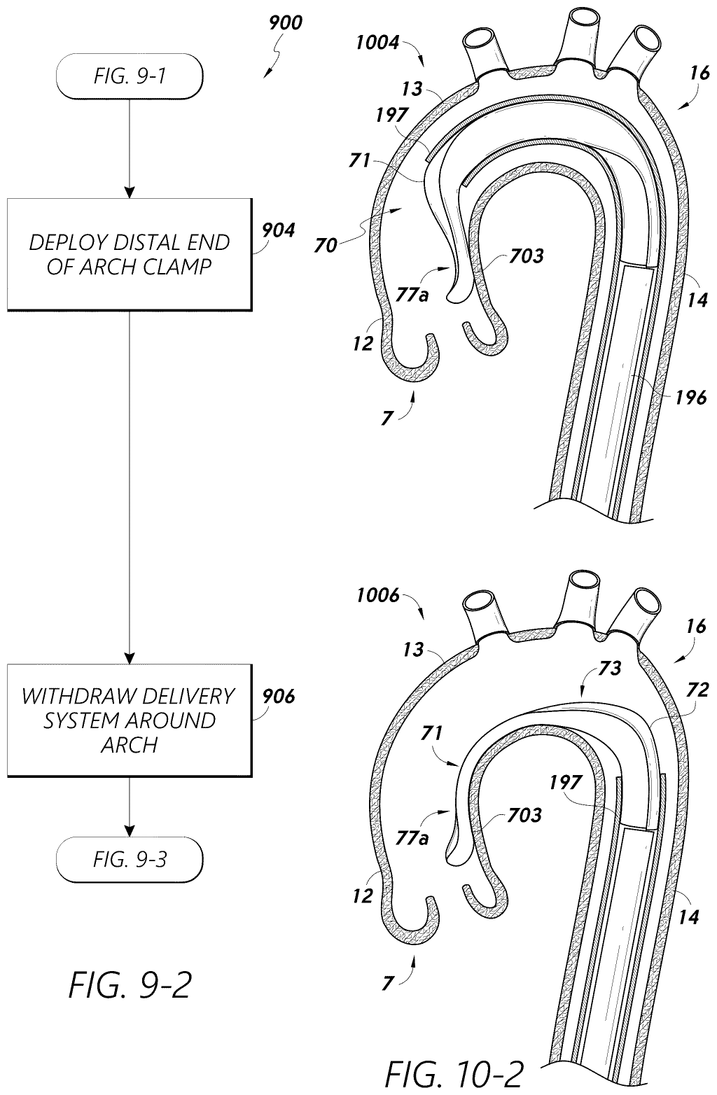

FIGS. 9-1, 9-2, and 9-3 illustrate a flow diagram for a process 900 for implanting a flow-leveling clamp device 70 in an aortic arch 13 in accordance with one or more examples. FIGS. 10-1, 10-2, and 10-3 provide images of the arch-clamp device 70 and certain anatomy corresponding to operations of the process of FIGS. 9-1, 9-2, and 9-3 according to one or more examples.

At block 902 (see also related images 1001, 1003), the process 900 involves advancing a delivery system 190 to a target position in a blood vessel 16 of a patient's vasculature, such as the aorta. For example, the delivery system 190 may be advanced through a percutaneous introducer or other minimally-invasive access 271 into the vasculature of the patient, and further within the vasculature to a target position within the aorta 16 of the patient. The delivery system 190 may include one or more catheters/sheaths 197 and/or a nosecone 199 or other feature configured to facilitate the forward/distal advancement of the delivery system 190 through tortuous anatomy of the vasculature. The percutaneous entry 271 may be at the femoral artery or other arterial blood vessel.

The delivery system 190 may have disposed therein an arch-clamp device 70, as described in detail herein. The device 70 may be disposed within the catheter/sheath 197 in an elongated delivery configuration, wherein the clamp form 705 is deformed to a straightened, elongated configuration, such that a natural/biased bend/fold portion 73 thereof is straightened to allow for the device 70 to assume a relatively low profile. The delivery configuration of the device 70 may further involve the lateral dimension LA (see FIG. 8) thereof being curved/wrapped around an inner diameter of the catheter/sheath 197. For example, while the clamp form 705 of the implant 70 may have a biased concavity as shown in FIG. 8, such concavity may be exaggerated and/or forced to a tighter curve in the delivery configuration shown in image 1003 to enable the small profile of the delivery configuration of the device 70 for fitting within the delivery catheter/sheath 197. In some implementations, in the delivery configuration, the clamp form 705 may be curved/wrapped laterally to a degree such that opposite lengthwise lateral edges of the form 705 laterally/circumferentially overlap and/or come into apposition or proximity with one another, as shown in image 1003.

At block 904 (see also related image 1004), the process 900 involves deploying a distal end 77a of the device 70 from the delivery catheter/sheath 197 in which the device 70 is held in the ascending aorta 12. As deployed, the distal end 77a of the implant 70 may be positioned on an inner radius 703 of the ascending aorta 12 and/or aortic arch 13 with respect to the curvature of the arch 13. With respect to description herein, the ascending portion of the aorta 16 may be considered any portion of the aorta that is on an upstream side of the inflection point of the arch 13, whereas the descending portion of the aorta 16 may be considered any portion of the aorta that is on a downstream side of the inflection point of the arch 13. Therefore, the aortic arch 13 may be considered to include portions of the ascending 12 and descending 14 aortas.

At block 906 (see also related image 1006), the process 900 involves proximally withdrawing the delivery system 190 around the arch 13 and back into the area of the descending aorta 14. At block 910 (see also related image (1010), the process 900 involves deploying the proximal portion/end 77b of the device 70 from the delivery system 190, such that a descending/proximal arm 72 of the clamp 70 is positioned in the descending aorta 14. Deployment of the implant 70 from the delivery catheter/sheath 197 may involve distally advancing a pusher device/structure 196 within the delivery catheter/sheath 197, and relative thereto, in a manner as to push the device 70 out from a distal end of the catheter/sheath 197. Additionally or alternatively, the catheter/sheaths 197 may be pulled proximally relative to the pusher 196 to deploy/expose implant 70.

As the implant 70 is deployed from the delivery system 190, it may assume a biased shape and/or configuration thereof. For example, a shape-memory of the device 70 may form a bend 73 in the arch 13 to thereby clamp the aortic arch to alter a geometry thereof as described herein. Furthermore, the lateral curvature of the device 70 may unfurl to some degree in a manner as to better conform to the radius of the target blood vessel. At block 912 (see also image 1012), the process 900 involves withdrawing the delivery system 190, thereby retaining the implant 70 in-place for the purpose of providing flow-leveling functionality in the target blood vessel to improve health prospects for the patient on an ongoing basis. For example, the ongoing operation of the implant 70 in the blood vessel 16 may operate in accordance with the cyclical transition between the configuration shown in FIG. 7-1 and the configuration shown in FIG. 7-2, described in detail above.

Vessel-Bending Stent/Anchor Assemblies with Bent Connecting Arms

As described in detail throughout the present disclosure, as individuals age, certain blood vessels can tend to become stiffer and, as a consequence, the vessels do not expand and contract, or “breathe,” as effectively with each beat of the heart. The result can be an increase in blood pressure and decrease in blood flow, particularly during systole. The cyclical expansion and compression of blood vessel volume, such as in the aorta, can help to modulate the rise in blood pressure. As described in detail above, the present disclosure provides various implantable devices configured to reshape a target blood vessel in a manner as to simulate natural compliance. Such devices/assemblies can include a shape-set wire mesh stent as a vessel-reshaping mechanism/framework. For example, to improve the vessel compliance and/or modulation of blood pressure, stent-like anchors may be implanted that are connected with a pre-shaped bridge component.

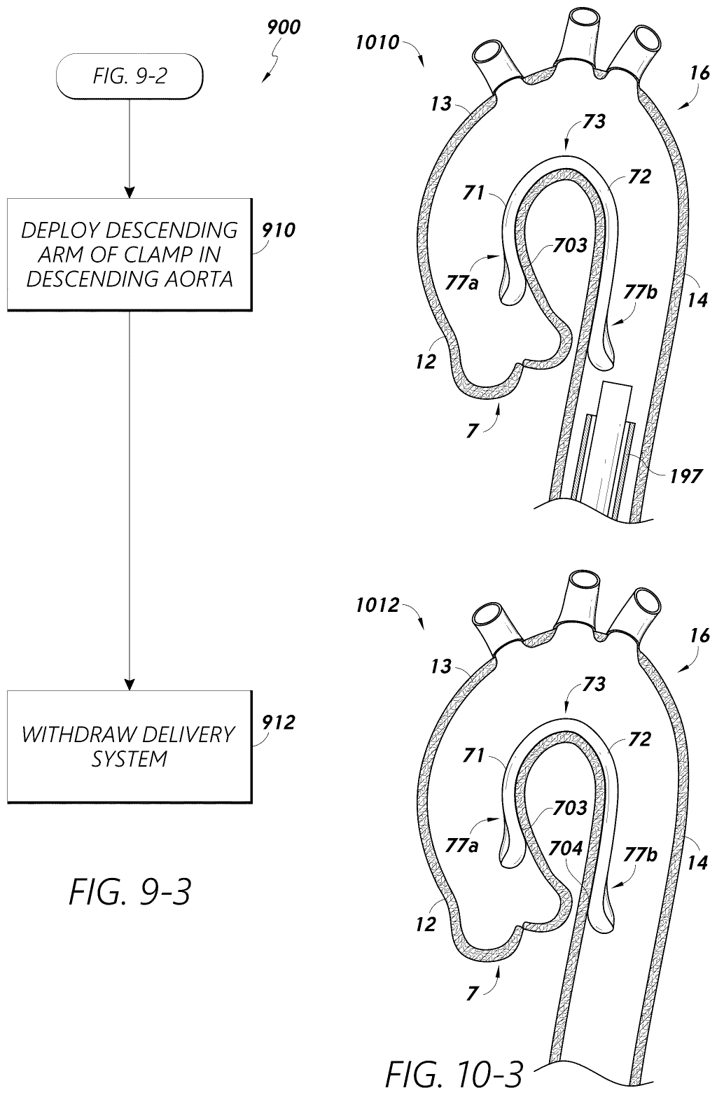

FIGS. 11A and 11B show views of a flow-leveling stent device/assembly 80 including a connecting arm/bridge 82 in accordance with one or more examples. In FIG. 11A, the device 80 is shown in a relaxed, bent configuration. FIG. 11B shows the device 80 with the connecting arm 82 straightened, such as by overcoming the spring force of the arm 82 that biases the arm in the bent/curved configuration.

The device/assembly 80 includes first Sla and second 81b stent anchors, which are shown in an expanded state/configuration, wherein the anchor devices 81 may be configured to be secured within a target blood vessel, such as a segment of an aorta. For example, the anchors 81 can advantageously have an expanded configuration with a diameter that is dimensioned to be approximately equal to, or slightly greater than, the diameter of the target blood vessel segment. The anchors 81 can advantageously be self-expanding, or may be balloon-expandable, or otherwise configurable for securing within a blood vessel. The device 80 can be pre-shaped in the bent configuration shown in FIG. 11A.

Two self-expandable stent/stent-like anchors 81 are physically connected by the shaped (e.g., bent/curved) arm/bridge 82. The entire implant 80 can be formed from a single laser-cut nitinol tube, or tube or sheet comprising other material. The device 80 can be implanted in a target blood vessel segment, wherein, when deployed/implanted, the device 80 can advantageously naturally bend and partially collapse the blood vessel during a low-pressure/relaxed state (e.g., diastole), wherein the vessel may be forced to at least partially straighten when the blood pressure increases (e.g., during systole). Therefore, the device 80 may cause bending and straightening of the blood vessel in a repeating cycle that produces fluid dynamics that mimic normal vessel compliance.

The connecting arm/bridge 82 is coupled between the first and second anchors 81. The arm 82, as with other features and devices disclosed herein, may have a biased bent/curved shape, such that in a relaxed/biased configuration as illustrated in FIG. 11A, the axes Aₛₐ and A_{sb} of the first 81a and second 801b anchors, respectively, are at a relative angle.

The connecting arm 82 may have any suitable or desirable shape or configuration. For example, in some implementations, the arm 82 includes a relatively narrow medial portion 83, wherein the arm 82 is relatively wider at the base portions 84a, 84b in the areas where the arm 82 couples to the anchors 81. Such narrow portion 83 may facilitate bending of the arm 82 and/or may accommodate the vascular anatomy and area where the target blood vessel is bent by reducing the area/volume of the arm 82 in such area, thereby reducing the physical contact area and/or obstruction of the arm. The narrow portion 83 may improve the flexibility in such segment of the arm 82, thereby accommodating the bend of the arm 82.

The arm 82 may be coupled to the anchors 81 and/or integrated therewith in some manner. For example, the anchors 81 may comprise stent frames cut from a tube, sheet, or other structure, such that the anchors 81 and arm 82 comprise a unitary, integrated form. In some implementations, attachment means, such as clips, sutures, hooks, clamps, or other fasteners, are used to attach the arm 82 to the anchors 81.

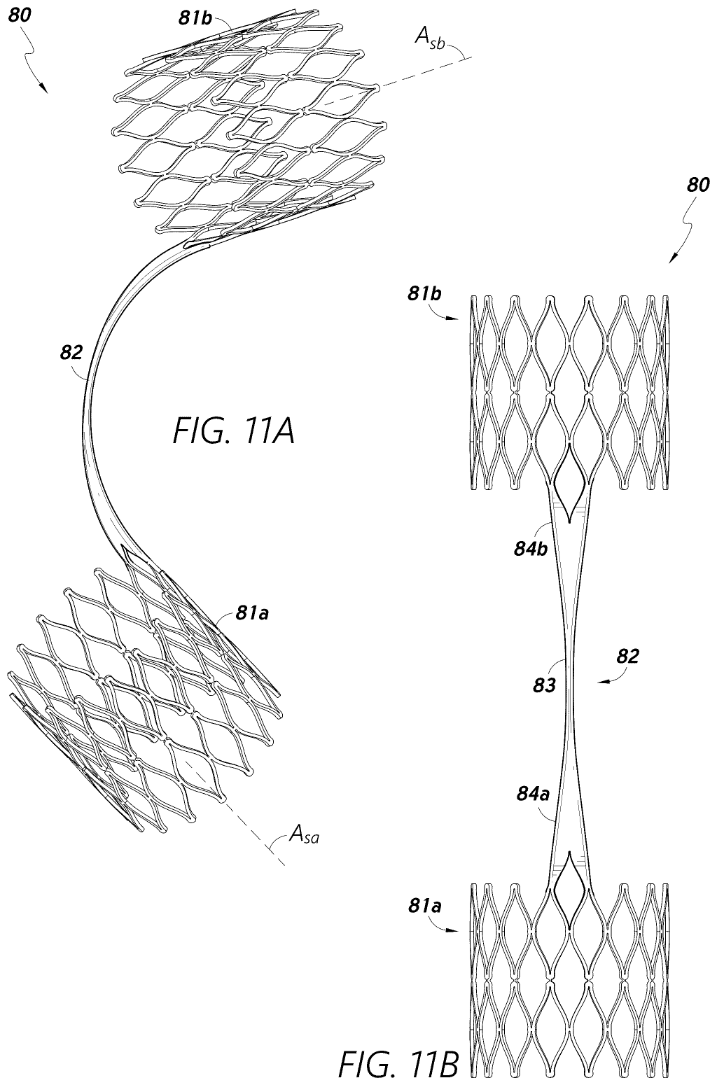

The flow-leveling device/assembly 80 may be implanted in a target blood vessel to implement cyclical, flow-leveling bending thereof as described in detail herein. FIGS. 12A and 12B show side cutaway and axial cross-sectional views, respectively, of a blood vessel segment 61 spanned by an implanted flow-leveling stent assembly 80 in accordance with one or more examples. The image of FIG. 12A shows the implant 80 implanted in the blood vessel 61 during a low-pressure cardiac phase (e.g., diastole), wherein the fluid pressure within the vessel 61 is sufficiently low to allow for the bending arm 82 to bend the blood vessel 61 to produce a tortuous path through the blood vessel 61 and/or cause radial compression of one or more dimensions of the blood vessel to produce a reduced volume thereof through reshaping.

With the blood vessel bent as shown in FIG. 12A, the cross-section of the blood vessel in an area of the bend 63 may have a non-circular shape, which can be desirable as allowing for volume change as the blood vessel alternates between non-circular and more-circular cross-sectional areas/volumes, as described in detail above. For example, the bending of the blood vessel 61 may cause the bend 63 to kink the blood vessel 61 to some degree, such as to produce the non-circular shape shown in FIG. 12B. With the blood vessel at least partially kinked, as shown, at least a portion of the blood vessel can provide an only-partially-open orifice shape during the diastolic phase due to the deformed/kinked blood vessel reducing blood flow.

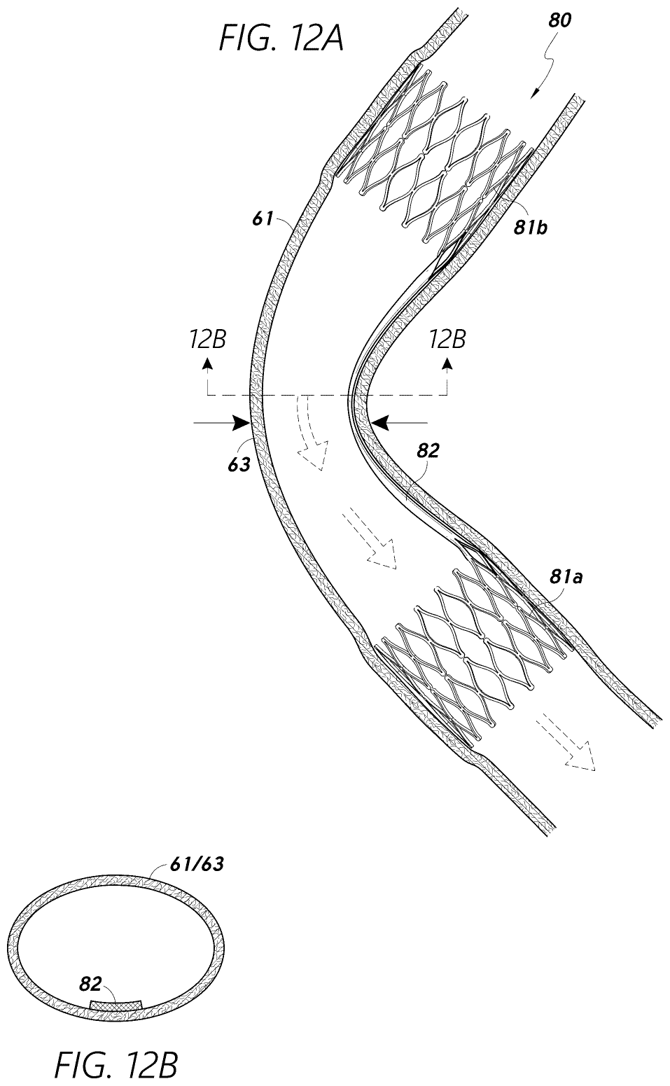

As the blood pressure increases in connection with higher-pressure phase(s) of the cardiac cycle, the pressure in the blood vessel 61 may overcome the spring force of the arm 82 to allow the arm 82 to be straightened and/or cause such straightening, as shown in FIG. 13A. FIGS. 13A and 13B show side cutaway and axial cross-sectional views, respectively, of the flow-leveling stent assembly 80 and blood vessel 61 of FIG. 12A being configured in a straightened configuration in accordance with one or more examples.

As the pressure increases, thereby causing the blood vessel to straighten and accordingly unbending the connecting arm 82, energy may be stored in the arm 82, such that when luminal pressures decrease, the energy can be returned to the circulation in the form of the arm 82 re-bending to the configuration shown in FIG. 12A. When straightened as shown in FIG. 13A, the blood vessel in the previously-bent segment 63 may return to a more-circular cross-sectional shape, as shown in FIG. 13B. In such configuration, the blood vessel may provide a more-open (e.g., fully-open) orifice, reducing flow resistance during systole. Therefore, as demonstrated in images of FIGS. 12A and 13A, the implant device/assembly 80 may be configured to cause cyclical bending and straightening of the blood vessel 61 in a manner as to decrease systolic flow and/or pressure and increase diastolic flow and/or pressure in the manner described in detail throughout the present disclosure.

Although a certain configuration of a pre-shaped, blood-vessel-bending implant device is shown in FIGS. 12A and 12B, it should be understood that blood-vessel-vending implant devices of the present disclosure may have various other structures. For example, such devices can be implanted in the aorta and perform re-shaping as shown and described herein.

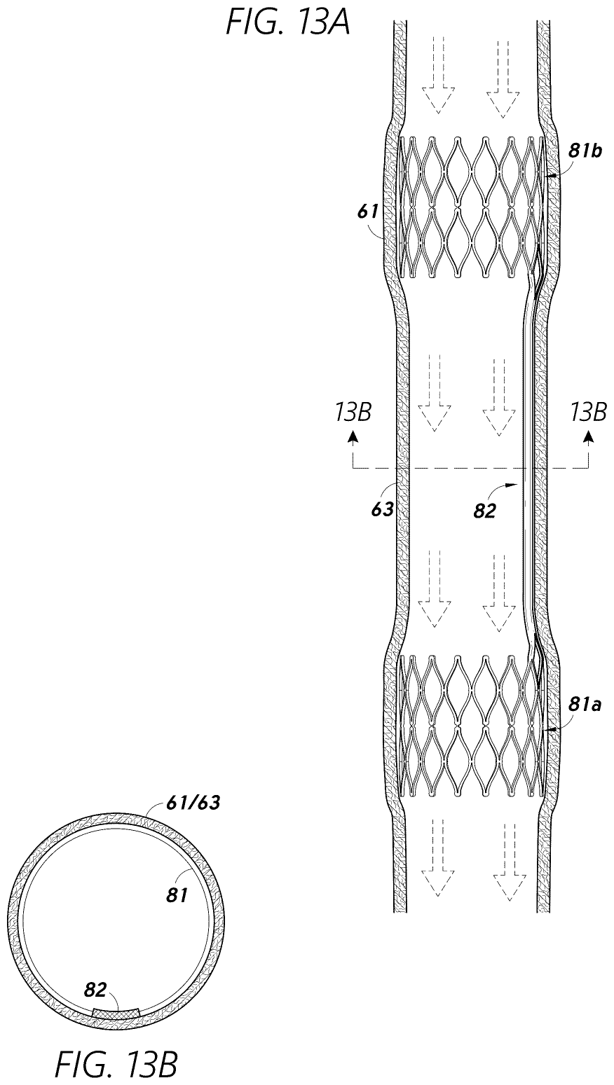

FIG. 14 shows a flow-leveling implant device/assembly 85 in a bent configuration in accordance with one or more examples. The device 85 may be similar in one or more respects to the device 80 described above. For example, the device 85 may include first 86a and second 86b anchors configured to anchor the device to first and second portions of a blood vessel, wherein such anchors 86 are connected by a connecting arm 87 that is biased in a bent/curved configuration, as shown.

The implant device 85 may include one or more stent-type anchors, as shown as the stent anchor 86a, for example. Furthermore, the device 85 may include one or more ‘C’-type/shaped anchors, such as the illustrated C-anchor 86b, which may be similar to the stent anchor 86a and/or may be distinguishable therefrom with respect to one or more features thereof. For example, the C-anchor 86b may comprise/form a circumferential opening 98, which may allow for the anchor 86b to be adjustable to accommodate different diameters and/or circumferences. The C-anchor 86b may have a solid, sheet-type form, as illustrated, or may include struts, cells, and/or other structural features or elements. The C-anchor 86b may be adjustable by a surgeon to adjust the diameter thereof in a manner as to produce a diameter that facilitates anchoring of the anchor 86b to the target blood vessel segment. For example, such adjustment may be made through bending the anchor 86b in a manner as to open and/or close the gap 98. A balloon or other instrument may be utilized to set the desired diameter of the C-anchor 86b.

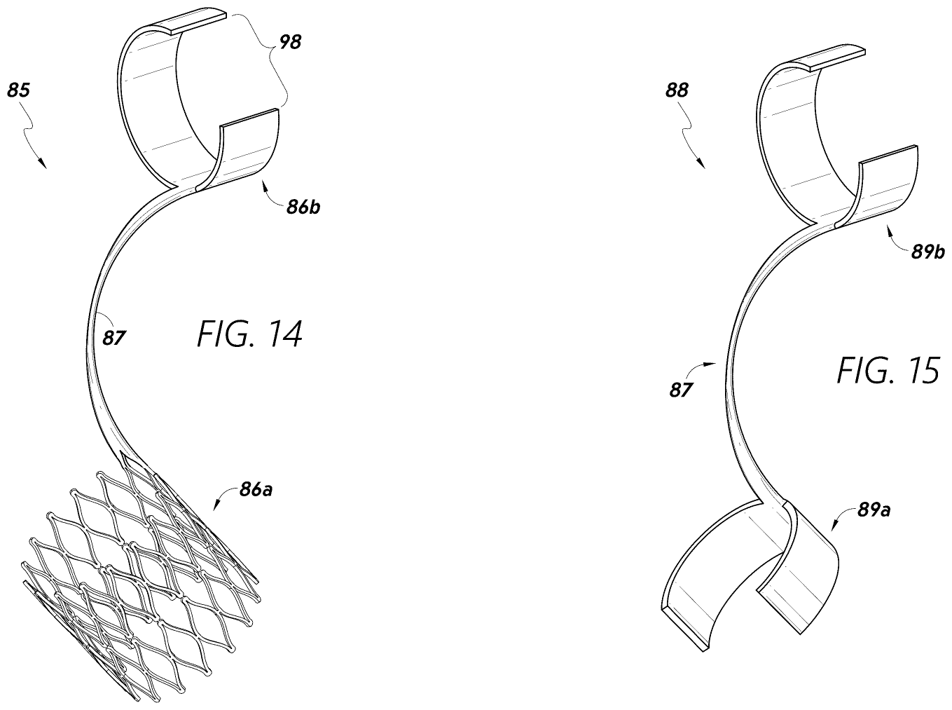

FIG. 15 shows a flow-leveling implant device 88 in a bent configuration in accordance with one or more examples. The device/assembly 88 includes first and second anchors 89a, 89b coupled by a connecting arm 87 that is biased to a bent and/or curved configuration, as described in detail herein. In the illustrated example of FIG. 15, the device/assembly 88 includes two C-type anchors, which may have any structure or configuration disclosed herein.

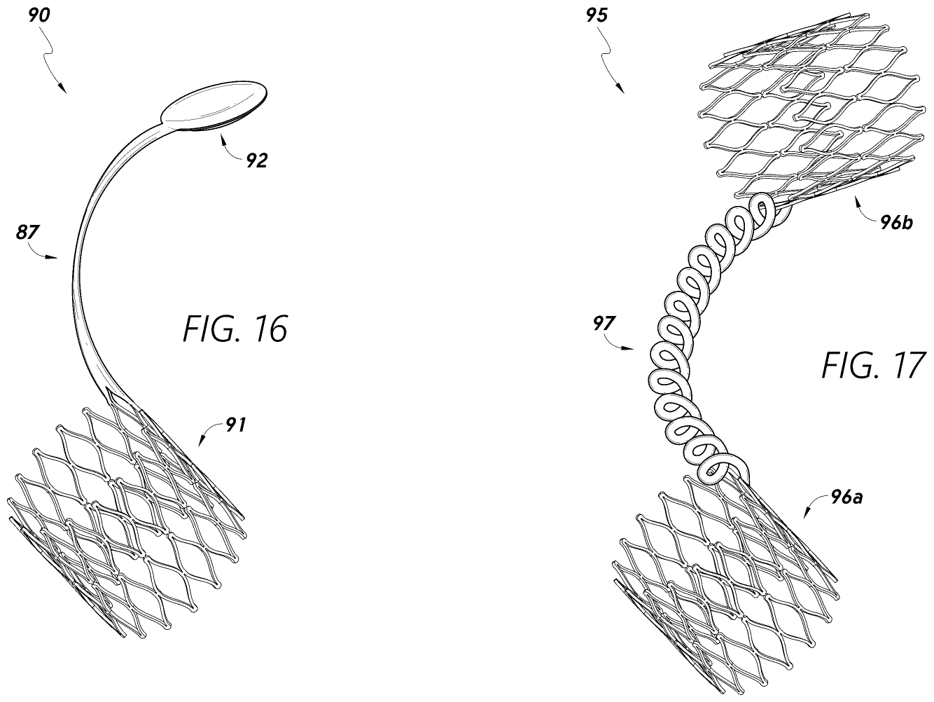

FIG. 16 shows a flow-leveling implant 90 comprising a contact pad 92, the implant being configured in a bent configuration in accordance with one or more examples. The implant/assembly 90 may be similar to any of the other with respect to anchors and or bent arms/features thereof. Example, the device 90 may include a first tissue anchor 91, which may have a stent-type form, or other blood vessel anchor form disclosed herein. The anchor 91 is coupled to a contact pad/structure 92 by a curved/bent connecting arm 87. While some implementations disclosed herein include two anchors connected by a connecting arm, it should be understood that some examples of the present disclosure may be implemented with a single anchor connected by a connecting arm to a contact pad, which may or may not have anchoring functionality/features. For example, the contact pad 92 may provide tissue contact that affords the implant 90 leverage for bending the target blood vessel segment using the connecting arm 87 by pressing against the blood vessel at the anchor 91 and the contact pad 92 to form a bend therebetween.

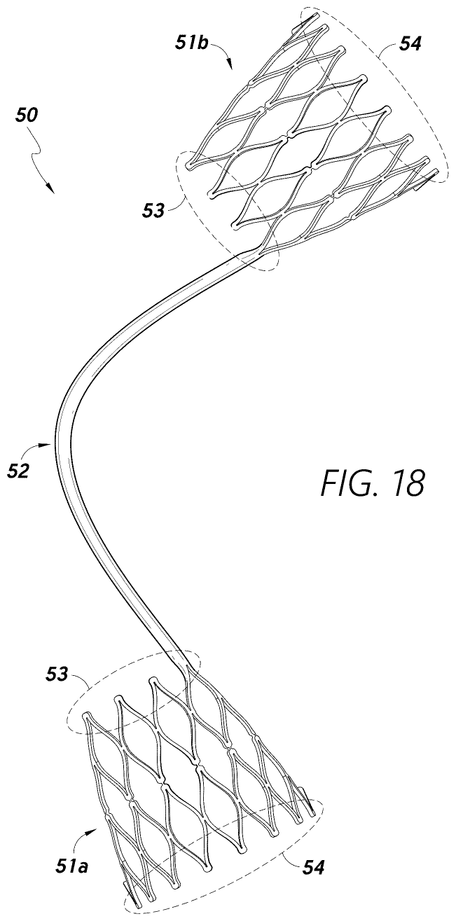

FIG. 17 shows a flow-leveling implant 95 comprising a coil/spring connecting arm 97 configured in a bent configuration in accordance with one or more examples. The device/assembly 95 may have first and second anchors and/or contact pad structures 96 connected by a coil-type connecting arm 97, which may be advantageous as allowing for the arm to have desirable flexibility for conforming with the desired bent shape of the blood vessel. Furthermore, the structure of the coil arm 97 may be relatively less traumatic to the blood vessel tissue relative to certain other arm configurations. The arm 97 may be biased in the illustrated bent/curved configuration, such that straightening of the anchors 96 and arm 97 may cause energy to be stored in the spring arm 97 that allows the blood vessel to be cyclically bent as pressure conditions change. The arm 97 may comprise a shape-set coil shape-set in the bent/curved configuration.

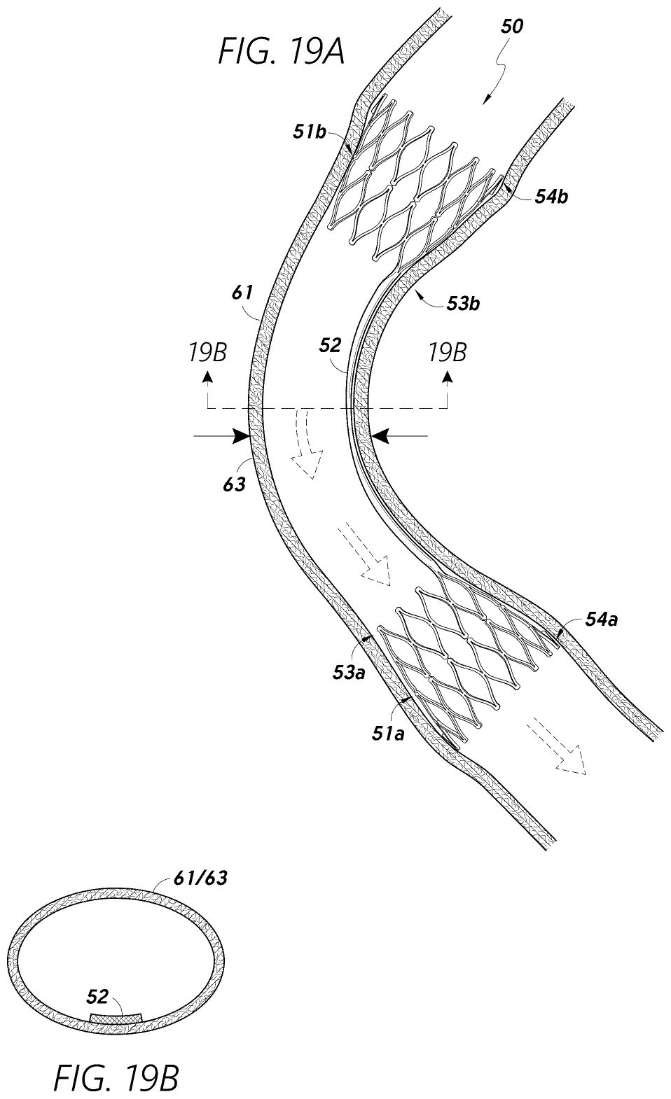

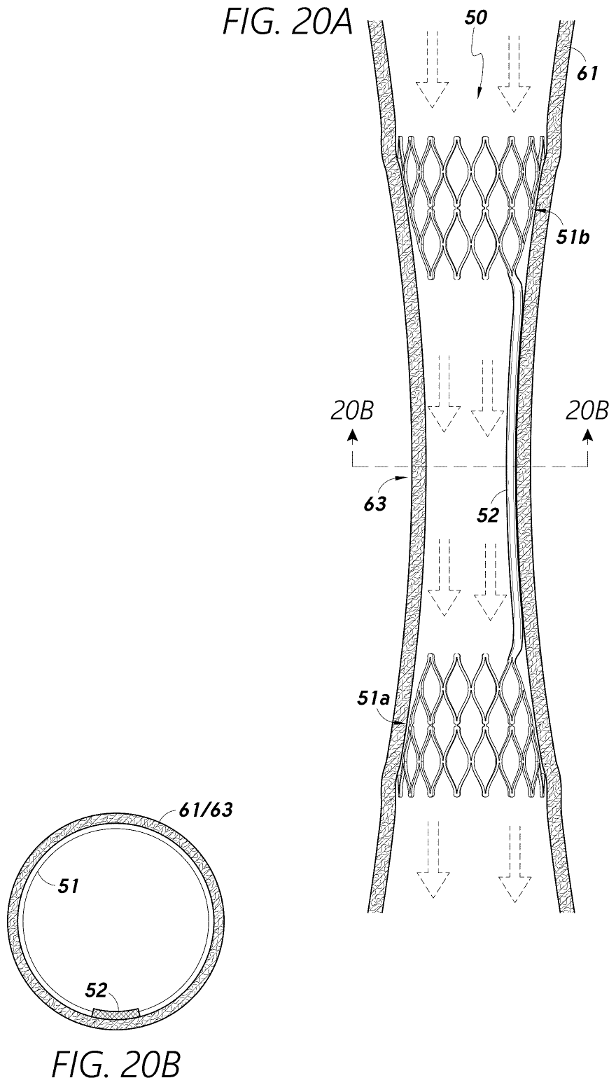

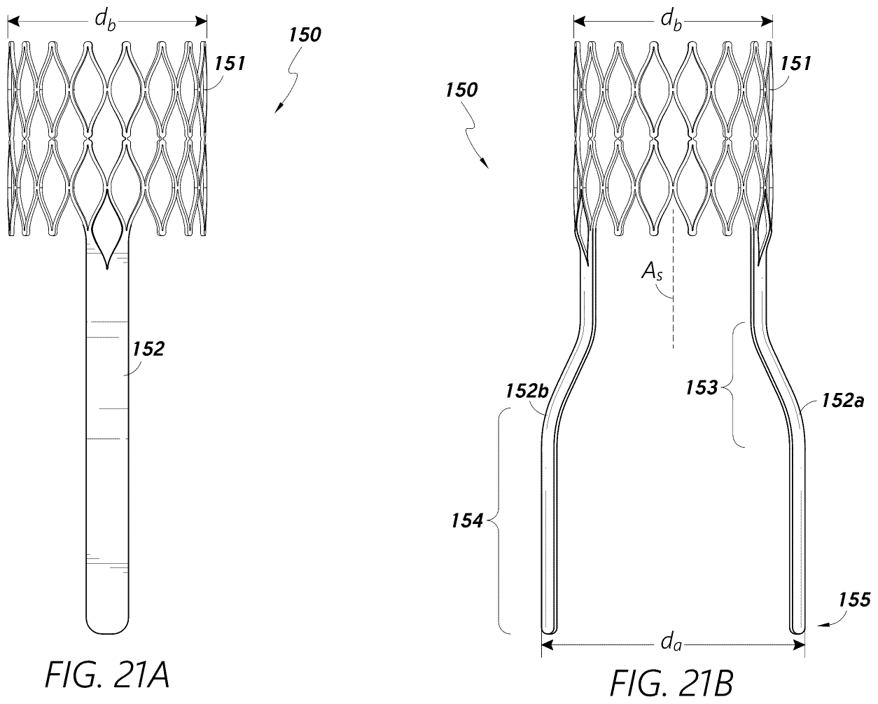

FIG. 18 shows a flow-leveling stent device/assembly 50 including one or more tapered stent anchors 51 connected by a connecting arm 52 in accordance with one or more examples. FIGS. 19A and 19B show side cutaway and axial cross-sectional views, respectively, of a blood vessel segment spanned by the implanted flow-leveling stent device/assembly 50 in accordance with one or more examples. FIGS. 20A and 20B show side cutaway and axial cross-sectional views, respectively, of the flow-leveling stent device/assembly 50, the flow-leveling stent device/assembly 50 being configured in a straightened configuration in accordance with one or more examples.

The anchors 51 may be tapered with a tapered end 53 having a diameter that is less than a wider end 54 of the respective tissue anchor 51. For example, the anchors 51 may be configured such that narrower tapered ends 53 thereof face towards an axial center of the device 50, as illustrated. The narrower diameter 53 of the anchors 51 may be implemented to accommodate the reshaping of a blood vessel segment by the device 50, wherein such reshaping may cause kinking/crimping of the blood vessel in the area of the bend therein. For example, as shown in FIG. 19A, the bend portion 63 of the blood vessel 61 may have a compressed diameter with respect to at least one dimension thereof as a result of the bending/curving of the blood vessel when the implant 50 is implanted, as illustrated. As described in detail above, such bending of the blood vessel can result in non-circular deformation of the blood vessel cross-section, as illustrated in FIG. 19B. Therefore, the narrower portions 53 of the anchors 51 may accommodate the compression of the blood vessel diameter in one or more dimensions, thereby reducing the impact/obstruction of the stent on the blood vessel and preventing the anchors 51 from interfering/obstructing with the bending of the blood vessel to an undesirable degree.

The wider ends 54 of the anchors 51 may be dimensioned to have a diameter greater than the natural diameter of the blood vessel in the implanted segment thereof to facilitate anchoring/securing of the anchors 51 to the blood vessel. The narrower diameter portions 53 of the anchors 51 may have a diameter that is approximately equal to the natural diameter of the native blood vessel, or slightly smaller than the natural diameter of the blood vessel. Such dimensions may advantageously provide desirable anchoring functionality, while reducing interference of the stents with the bending of the blood vessel.

FIG. 20A shows the blood vessel 61 in the straightened, high-pressure configuration, wherein the biasing of the connecting arm 52 has been overcome to straighten device 50, in a similar manner as with other examples disclosed herein.

Stent Devices with Vessel-Reshaping/Ovalizing Arms

As described in detail above, a blood vessels age, such as the aorta, The blood vessel tissue can become stiffer and fail to contract and expand as efficiently with each heartbeat as with healthy, compliant blood vessels. This stiffening of the blood vessel tissue and associated reduced compliance can result in increased blood pressure and/or lower blood flow. The compliance of relatively large blood vessels, such as the aorta, is particularly helpful for moderating cyclical rises in blood pressure. In some implementations, examples of the present disclosure can include stent implant devices that, when implanted, are configured to decrease the cross-sectional area/volume of the blood vessel segment in which the stent is implanted during low-pressure conditions, such as diastole, which serves to force blood through the blood vessel segment by pushing the blood through the vessel as the vessel volume reduces in connection with stent contraction induced by cyclical drops in blood pressure. In some implementations, blood-vessel-reshaping stent implant devices of the present disclosure comprise certain wire-frame stent and shape-biased arm features/components.

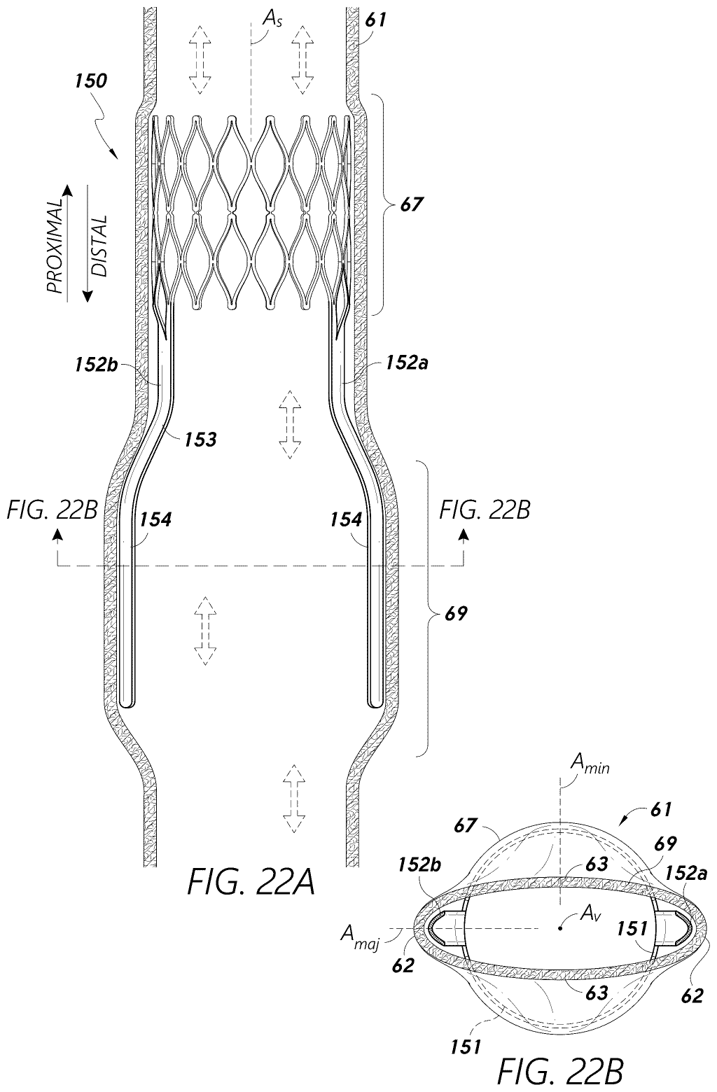

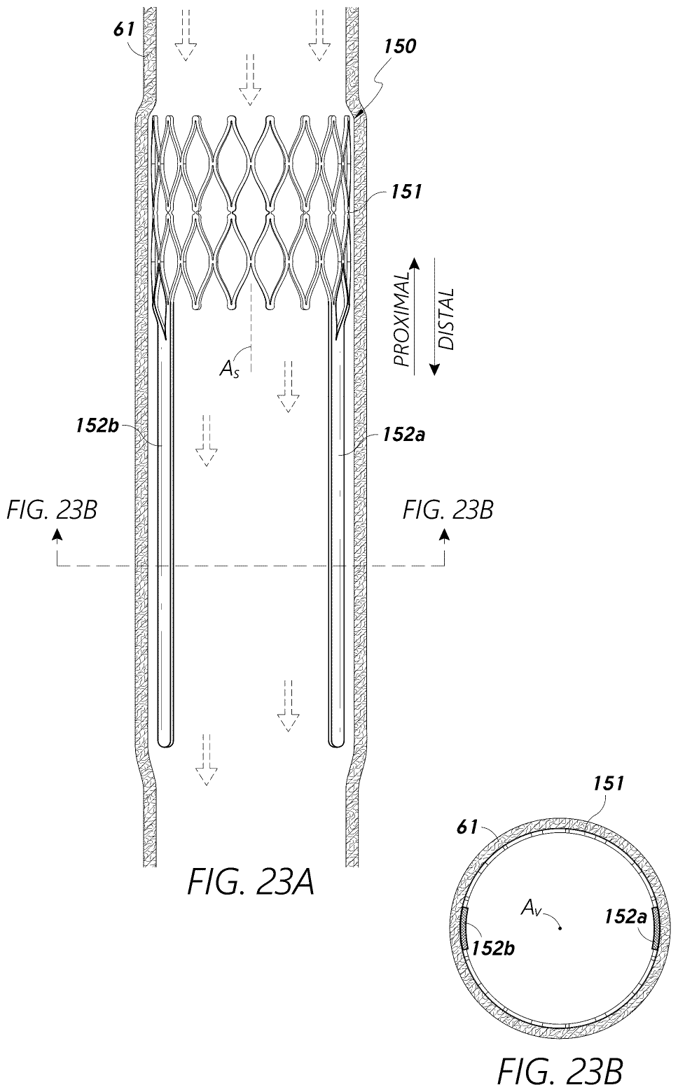

FIGS. 21A and 21B show views of a flow-leveling stent 150 including ovalizing arms 152 in accordance with one or more examples. FIGS. 22A and 22B show side cutaway and axial cross-sectional views, respectively, of the flow-leveling stent 150 implanted in an example blood vessel segment 61. The arms 152 of the implant 150 emanate from, and/or are coupled to, an anchor 151 (e.g., stent segment) configured to be expanded and secured within the blood vessel segment 61 to hold the implant 150 in-place therein. Compared to stent implants including fully-circumferential stent frames configured to form an oval shape to thereby ovalize a target blood vessel segment, the implant 150 and other implants including ovalizing arms that extend axially from a stent or other anchor can be relatively minimalistic, at least with respect to ovalizing segments thereof. Therefore, implants including ovalizing arms, such as the implant 150, can produce blood vessel re-shaping functionality with reduced structural bulkiness and/or reduced blood flow impedance.

The arms 152 extend/project from one axial end of the stent/anchor frame 151, as shown. The arms 152 may be an integrated form with the stent frame 151. The arms 152 project in a distal direction from the frame 151. The arms 151 may terminate at distal ends thereof in free ends 155.

The implant arms 152 can be used to leverage the aspect of tubular blood vessels generally having a maximum cross-sectional area and volume for a given perimeter when the cross-sectional area thereof is that of a circle, and therefore deviations from the circular shape produce a decrease in area/volume relative to the circular shape. To the extent that a blood vessel is noncompliant, the vessel may have a relatively fixed circumference. Therefore, forcing a non-compliant blood vessel into a non-circular cross-sectional shape can result in the volume of blood flowing through the blood vessel being forced through a relatively smaller volume, thereby exerting increased pressure on the blood flow. The implant arms 152, in a biased state thereof, are configured to reshape the blood vessel 61 to a non-circular shape, wherein pressure increase in the blood vessel 61 (e.g., during systole) causes the hoop stress in the blood vessel wall to overcome the biased shape of the arms 152, thereby straightening the arms and allowing the blood vessel to become more circular.

The stent portion/segment 151 of the implant 150 can include a wire-frame stent or anchor. For example, the stent 151 may be similar to any stent devices disclosed herein. The arms 152 can be positioned on an upstream or downstream side (shown on the downstream side in FIG. 22A) of the stent 151. The arms 152 may be coupled to the stent/anchors 151 and/or integrated therewith in some manner. For example, the anchor 151 may comprise a stent frame cut from a tube, sheet, or other structure, such that the anchor 151 and arms 152 comprise a unitary, integrated form. In some implementations, attachment means, such as clips, sutures, hooks, clamps, or other fasteners, are used to attach the arms 152 to the anchor/stent 151.

The arms 152 can comprise spring projections/extensions from the anchor 151, wherein the arms 152 have an outward bias with respect to the axis Aₛ of the anchor/stent 151. The outward bias, as shown in FIGS. 22A and 22B, causes the segment 69 of the vessel 61 spanned by the outwardly-deflected arm portions 154 to take on an oval cross-sectional shape. The arms 152 may advantageously emanate from opposite circumferential/diametrical sides of the stent/anchor portion 151, as shown. Although two arms 152 are shown, it should be understood that more than two ovalizing arms may extend from the stent 151. The arms 152 may each terminate in respective free ends 155, which may have certain atraumatic features, such as curved surfaces, or the like. For example, the free ends 155 may deflect/project radially inwardly to avoid scraping/abrading the inner blood vessel wall.

The stent/anchor 151 can have a fluid-tight or fluid-impeding covering/cover on an inner and/or outer diameter thereof. Coverings and other components of example implant devices of the present disclosure can comprise any type of biocompatible material, such as, but not limited to, expanded polytetrafluoroethylene (ePTFE), polyester, polyurethane, fluoropolymers (e.g., perfluoroelastomers and the like), polytetrafluoroethylene, polyethylene terephthalate (Dacron), silicones, urethanes, ultra-high molecular weight polyethylene, aramid fibers, and combinations thereof.

The arms 152 are configured to generate a differential cross-sectional area or volume of the target blood vessel 61 (e.g., aorta) between high- and low-pressure phases of the cardiac cycle to facilitate perfusion. As described above, relatively non-compliant blood vessels generally may not be able to stretch to thereby lengthen the perimeter of the blood vessel in response to increased pressure conditions. Such inability to stretch can prevent compliant expansion of the blood vessel. The implant 150 produces complaint blood vessel volume change by manipulating/reshaping the native blood vessel walls 61, thereby increasing compliance in the blood vessel 61 without requiring blood vessel grafting or resection. Therefore, compared to blood flow solutions involving blood vessel grafting/resection, examples of the present disclosure can provide a solution that avoids the risks that may be associated with cutting of the vessel and/or devices grafted in/to such vessels, which may present risk of rupture and blood leakage outside of the circulatory system. Hazards associated with extravascular arterial blood leakage, such as within the abdominal and/or chest cavity, can include the risk of serious injury or death.

As described above, desirable diastolic flow in arterial (or venous) blood vessels is enabled by the decrease in cross-sectional area/volume of the blood vessels when transitioning from higher-pressure conditions (e.g., systole) to lower-pressure conditions (e.g., diastole). Where the relevant blood vessel has become stiff and non-compliant, stretching/expanding and subsequent contraction/shrinking of the blood vessel to cause the desired change in area/volume of the blood vessel may be limited due to the perimeter/wall of the blood vessel being resistant to stretching. Examples of the present disclosure provide implants that cause a change in cross-sectional area/volume of a target blood vessel without requiring stretching in the blood vessel wall by manipulating the shape (e.g., cross-sectional shape) of the target blood vessel; the transition between blood vessel shapes occurs in response to changing pressure conditions can reduce and increase the area/volume of the blood vessel in a cyclical manner to promote more even flow of blood through the blood vessel throughout the cardiac cycle.

Examples of the present disclosure provide for stent-type implants that include vessel-reshaping arms that are biased to a wider diameter compared to the diameter of the stent/anchor segment(s) from which the arms emanate. With respect to the implant 150, in a relaxed/non-pressurized state, a first diameter dₐ of the arms 152 has a greater dimension compared to a diameter d_{b} of the stent/anchor 151, wherein the arms 152 are configured to transition to a straighter, reduced diameter (e.g., diameter d_{b}) when pressure within the blood vessel 61 overcomes the expanded/deflected bias of the arms 152 and allows for the blood vessel segment 69 to revert to a more-circular shape. The ability of some implant devices of the present disclosure to reshape a target blood vessel in the manner described above to produce the desired oval cross-section (see FIG. 22B) of the blood vessel 61 can be achievable due to stiff/non-compliant blood vessels, which may be unable to stretch to a substantial degree, still retaining the ability to bend to a sufficient degree to allow for such shaping of the blood vessel. That is, the bending stiffness of a non-compliant blood vessel may be relatively lower compared to the stretching stiffness thereof. Therefore, examples of the present disclosure achieve compliance through bending energy with respect to the blood vessel wall, as opposed to stretching energy.

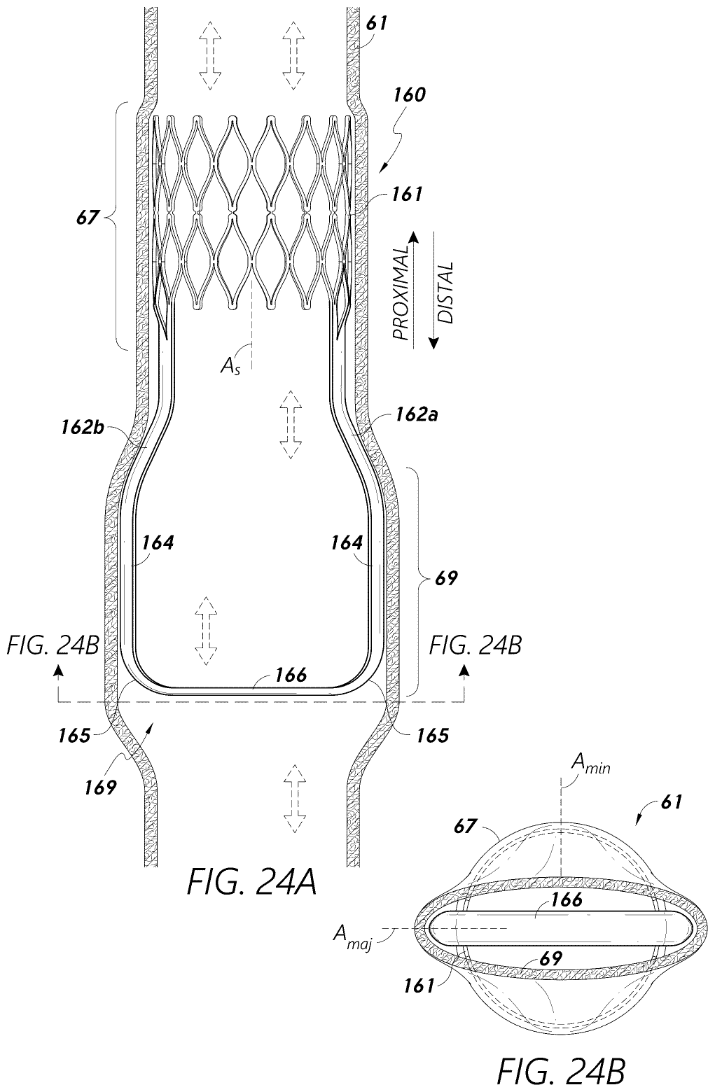

FIGS. 23A and 23B show side cutaway and axial cross-sectional views, respectively, of the flow-leveling implant 150 with the arms 152 in a straightened configuration in a high-pressure condition in accordance with one or more examples. Though the arms 152 of the implant 150 may be biased toward the outwardly-deflected configuration shown in FIGS. 21A, 21B, 22A, and 22B, they may be configured to, when subjected to mechanical forces associated with high luminal pressure, responsively transform to a more-straightened systolic configuration (see FIGS. 23A, 23B).

During systole, pressure through the oval cross-sectional shape formed by the arms 152 causes pressure to increase, which may force the area of the vessel farthest from the arms 152 (e.g., in the minor-axis Aₘᵢₙ area of the ovalized blood vessel segment 69) to push outward, overcoming the spring force of the arms 152, and bringing them inward against the spring bias. This in turn causes the vessel 61 to take on a rounder cross-sectional shape, thereby creating compliance. When the vessel-reshaping arms 152 are forced to a straighter/narrower configuration (with respect to the axial dimension of the anchor 151), energy may be stored in the shape memory of the arms 152, such as in the deflection/transition portions 153 thereof, wherein recoil/expansion of the arms 152 towards their biased, ovalizing/expanded configuration can return/release energy to the blood circulation. When the arms 152 are straightened, they may generally be more in-line/parallel with the axis Aₛ of the anchor/stent 151.

As shown in FIGS. 23A and 23B, luminal pressure forces against the blood vessel wall 61 increase the hoop stress on the blood vessel, which may force the blood vessel, and with it the arms 152, to conform to a more-circular shape. The resulting hoop stress, also referred to as “tangential stress” or “circumferential stress,” from luminal pressure increase exerts radially-outward force along the blood vessel's inner circumference, such stresses/forces being tensile in nature, which can tend to cause the blood vessel to increase in diameter. However, where the elasticity of the blood vessel wall is compromised, as with the blood vessel 61, the expansion of the blood vessel diameter is limited, and therefore, the pressure increase reshapes the blood vessel without substantially increasing the circumference thereof. The blood pressure force on the blood vessel wall and resulting inward deflection of the major-axis blood vessel walls (due to outward deflection of the vessel wall portions in the area of the minor axis Amin) may cause inward deflection of the arms 152 to form a desired geometric change to conform to a more-circular shape of the anchor/stent 151 and blood vessel 61.

With respect to implantation within the aorta or other arterial blood vessel, the systolic phase of the cardiac cycle, during which pressure levels in the aorta/arteries are relatively higher, causes the expansion of the blood vessel 61 and resulting inward deflection of the arms 152 to conform to the more-circular shape (shown in FIG. 23B), whereas the diastolic phase, which is associated with relatively lower arterial blood pressure levels, allows for the expansion of the arms 152 in the major axis Aₘₐⱼ dimension to the lower-energy ovalizing configuration, thereby forcing the blood vessel 61 to assume a more oval shape.

With reference to the axial view of FIG. 22B, as the spring arms 152 push outward on the major-axis walls/portions 62 of the blood vessel 61, the minor-axis walls/portions 63 of the blood vessel 61 may be pulled/drawn at least partially towards an axial center Aᵥ of the blood vessel 61 and/or towards each other in a manner as to cause the blood vessel 61 to form a non-circular/non-cylindrical shape, such as an oval shape as shown in FIG. 22B. That is, compared to the circular/cylindrical shape of the blood vessel 61, the blood vessel 61 in the oval shape has a cross-sectional area in the axial segment/area where the deflected portions 154 of the arms 152 are implanted that is reduced.

As blood pressure increases, the luminal pressure in the area 69 of the blood vessel 61 can exert radial outward force against the vessel internal wall, wherein such forces indirectly act against the arms 152 of the implant 150 to allow for outward deflection of the minor-axis walls/portions 63 of the vessel towards the circular shape of the blood vessel segment 67. For example, as the blood pressure increases in the vessel 61, the hoop stress on the blood vessel walls may force the side wall portions 63 of the blood vessel 61 to deflected radially outward towards a more uniform circular shape of the blood vessel 61, wherein the stiffness of the blood vessel wall causes the outward deflection of the sidewalls 63 to pull radially inward on the end walls 62 of the blood vessel, thereby applying radially-inward pressure on the arms 152 of the implant 150.

As the pressure in the blood vessel 61 increases (e.g., in connection with the systolic phase of the cardiac cycle), the plastically-deformable nature of the spring arms 152 allows for the arms 152 to be deformed/remodeled to the straighter and/or narrower configuration to accommodate the shortening of the major axis dimension Aₘₐⱼ of the blood vessel segment 69. The implant arms 152 improve cardiac perfusion by causing a decrease in cross-sectional blood vessel area during diastole relative to systole due to the reshaping of the blood vessel 61 caused by the lengthening of the major diameter Aₘₐⱼ of the blood vessel 6.

The anchor/stent 151 and/or arms 152 may be inclined to experience tissue in-growth in one or more areas thereof. As with any of the examples disclosed herein, the implant arms 152 can be configured to deform/transition from the ovalizing outwardly-deflected configuration of FIGS. 21A/B and 22A/B to the more-circular shape of FIGS. 23A/B in the presence of threshold blood pressure levels greater than 80 mmHg, such as blood pressure levels greater than 90 mmHg (e.g., between 90-120 mmHg).

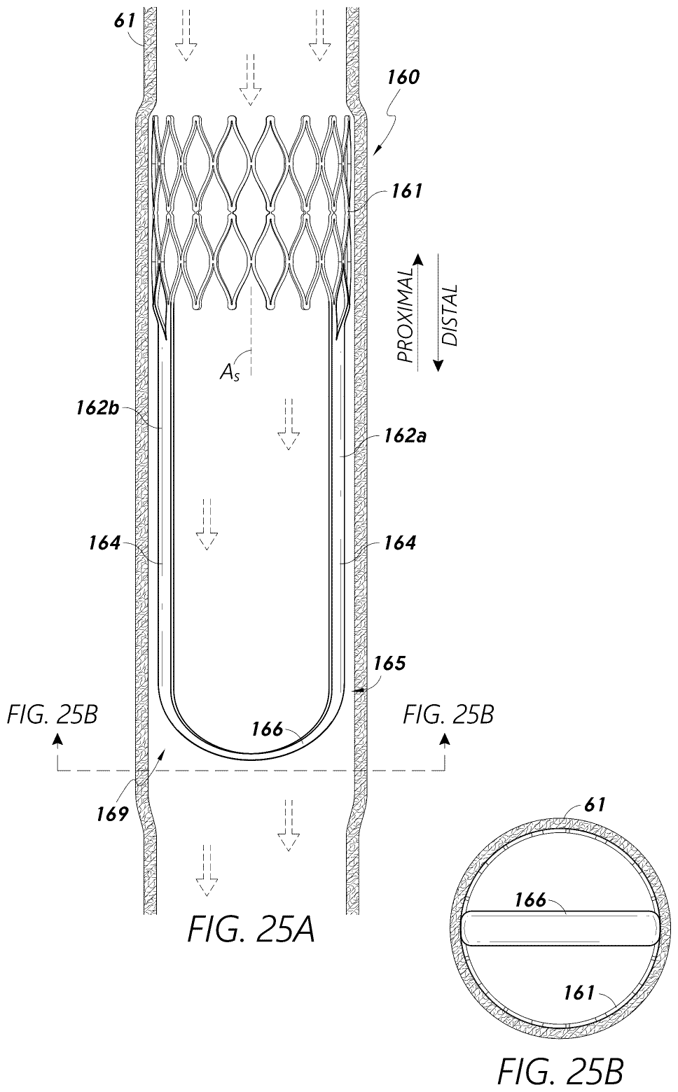

FIGS. 24A and 24B show side cutaway and axial cross-sectional views, respectively, of a flow-leveling implant 160 including joined ovalizing arms 162 implanted in a blood vessel segment 61 in accordance with one or more examples. The implant 160 may be similar in one or more respects to the implant 150 described above. However, the arms 162a, 162b of the implant 160, which are configured to deflect radially outwardly with respect to the axis Aₛ of the anchor/stent component 161 of the implant 160 so as to ovalize the blood vessel segment 69, may be joined, coupled, or otherwise integrate in some manner, rather than terminating in respective free ends.

In some examples, the arm portions 162 may be coupled by a crossbar connector 166, which may pass diametrically across the diameter of the blood vessel 61 and/or stent 161. For example, the crossbar 166, in the expanded/deflected biased configuration shown in FIGS. 24A and 24B, may extend along the major axis dimension Aₘₐⱼ of the oval portion 69 of the blood vessel 61 when the arms 162 are expanded/deflected to the ovalizing configuration shown in FIGS. 24A and 24B. The arm portions 162a, 162b and the crossbar 166 may be formed of a single integrated bar/arm, which may form a closed loop as shown in FIG. 24A.

The crossbar 166 is coupled to and/or emanates from distal ends/end-portions of the arms 162. The crossbar 166 can be considered to be disposed at a distal end/end-portion of the implant 160. The crossbar 166 crosses/spans across the diameter of the stent frame 161 and/or blood vessel 61. The arms 162a, 162b and the crossbar 166 can be formed of a single bar/arm structure, which may form a closed loop with the stent frame 161 with respect to the side view shown in FIG. 24A.

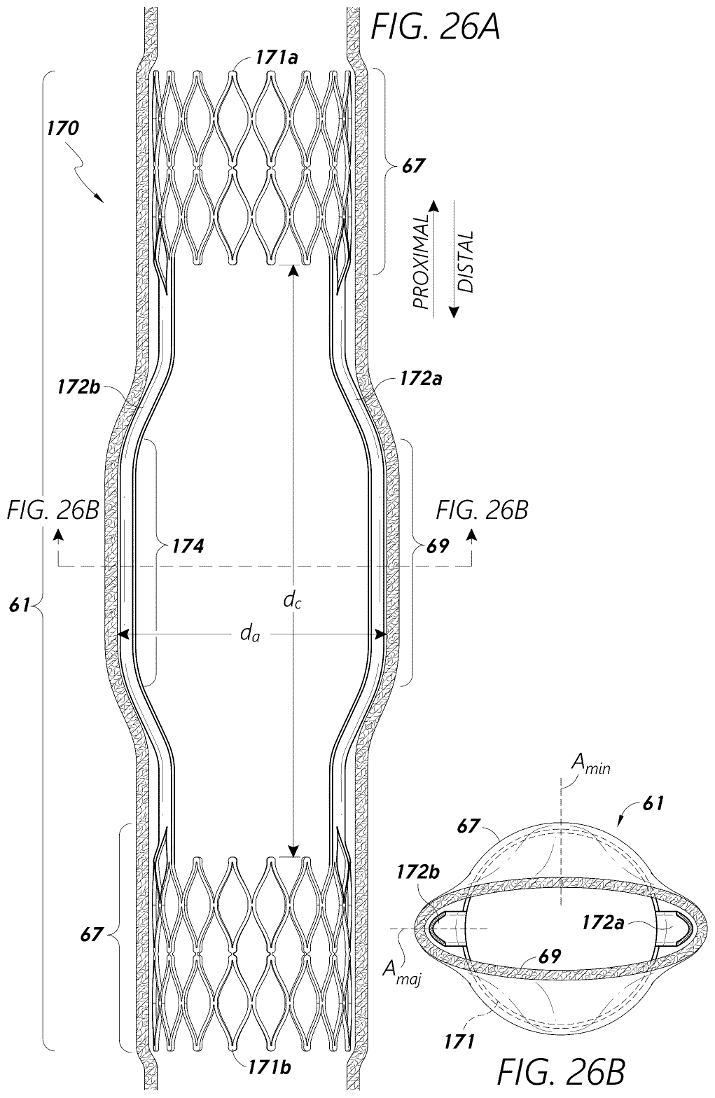

In response to increased hoop stress on the blood vessel wall resulting from increased luminal pressure (e.g., during systole or other high-pressure condition), the arms 162, and/or expanded/deflected portions 164 thereof, may be forced inward to a straighter and/or narrower configuration, as shown in FIGS. 25A and 25B. FIGS. 25A and 25B show side cutaway and axial cross-sectional views, respectively, of the flow-leveling implant 160 configured with the arms thereof in a straightened configuration in accordance with one or more examples.

When the arms 162 are deformed to the straighter configuration, the crossbar 166 may necessarily become (more) axially bowed-out, as shown in FIG. 25A. The crossbar 166 may advantageously be configured to bend/curve as necessary to accommodate circularization of the blood vessel 61, while maintaining some amount of structural rigidity/stability to support the arms 162 in their expanded configuration and/or to prevent further inward collapsing of the arms 162 in the deformed configuration of FIG. 25A. The crossbar 166 may serve to hold the deflected portions 164 of the arms 162 in the expanded configuration of FIG. 24A in low-pressure conditions to contribute to the reshaping of the blood vessel portion 69 by pushing/holding the arm portions 164 in the expanded-diameter configuration shown in FIGS. 24A and 24B. Furthermore, the crossbar connection 166 between the arms 162 can produce a desirably atraumatic contact between the arms 162 and the blood vessel wall. For example, whereas free ends of implant arms as disclosed herein, in some cases, can cause abrasions or other contact/damage with the tissue wall during implantation and/or cardiac cycling, the inherent curved bends 165 can present contact surfaces the are sufficiently atraumatic to prevent or reduce risk of damage to the blood vessel wall by the arms 162.

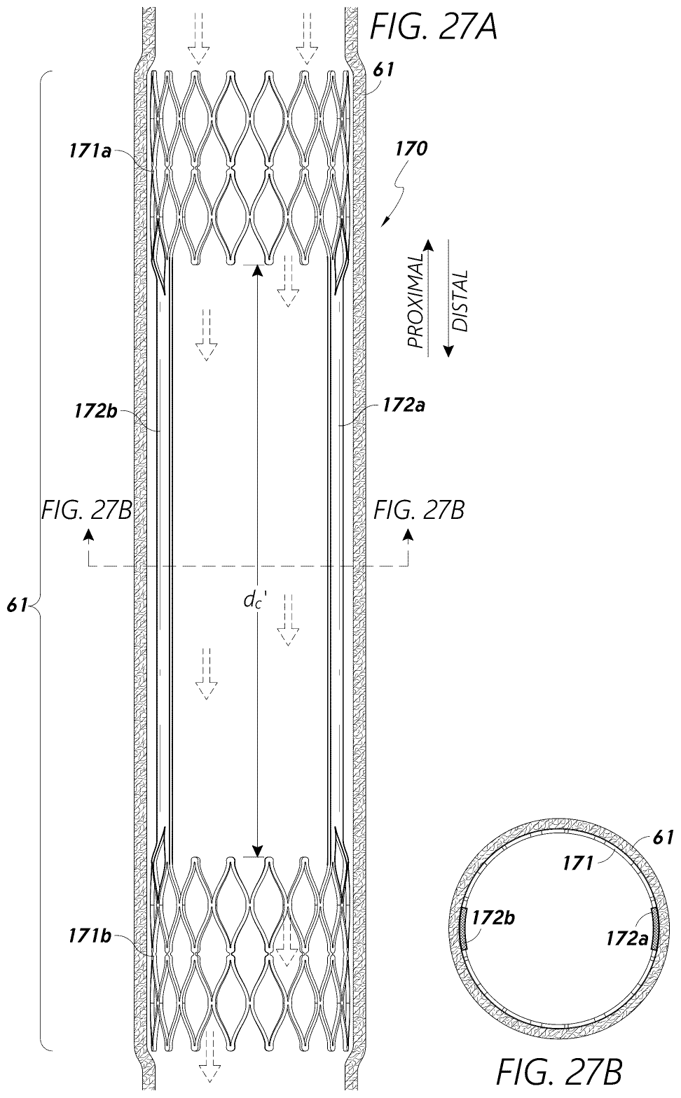

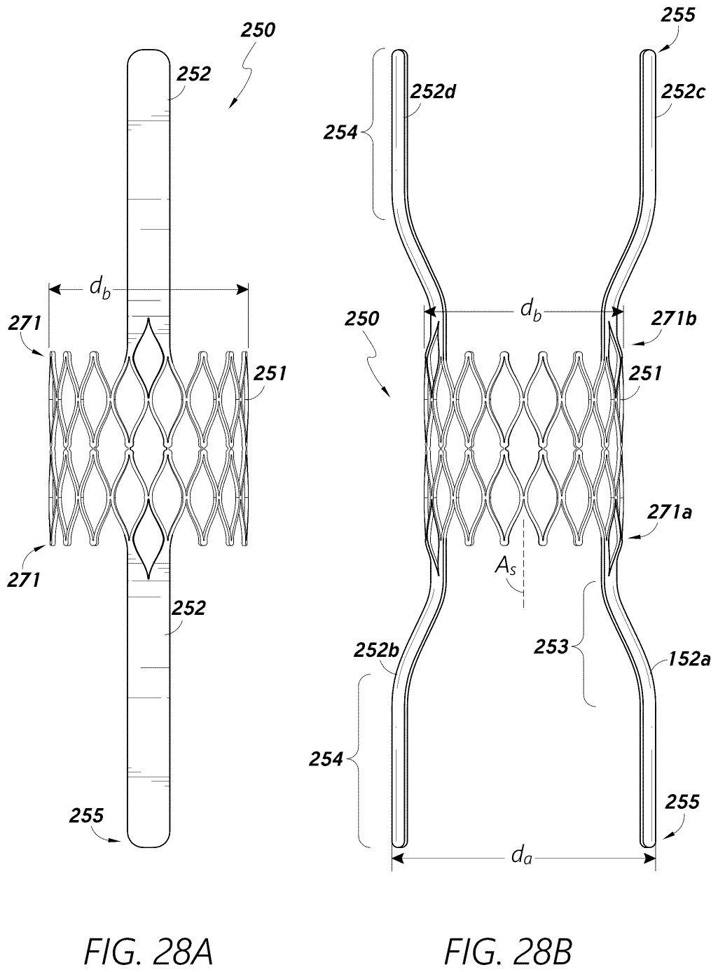

FIGS. 26A and 26B show side cutaway and axial cross-sectional views, respectively, of a flow-leveling stent assembly/implant 170 including stent/anchor segments 171 coupled by ovalizing arms 172 implanted in a blood vessel segment 61 in accordance with one or more examples. FIGS. 27A and 27B show side cutaway and axial cross-sectional views, respectively, of the flow-leveling stent assembly/implant 170 configured with the arms 172 thereof in a straightened/narrowed configuration in accordance with one or more examples. The implant 170 may be similar in any respect to any of the other examples of implant devices including blood-vessel-ovalizing arms disclosed herein.

The implant 170 may advantageously include multiple stent segments 171, such as the illustrated stent segments 171a and 171b, wherein the stent/anchor segments 171 may be axially offset from one another and connected by connector arms 172. For example, two or more anchor arms may span the axial distance d_{c} between the stent segments 171a, 171b. In the illustrated example, first 172a and second 172b arms extend between opposite sides of the stents/anchors 171. The arms 172 may deflect radially outwardly, as with other examples disclosed herein. For example, at least an axial segment 174 of the arms may produce an expanded ovalizing major-axis diameter dₐ, which in turn produces an expanded major-axis Aₘₐⱼ dimension of the blood vessel segment 69 in which the expanded arm portions 174 are disposed.

In high pressure conditions, as shown in FIGS. 27A and 27B, the arms 172 may straightened-out, thereby allowing the blood vessel 61 to assume a more-circular configuration, as shown in FIG. 27B. The straightening/narrowing of the arms 172 may cause some amount of axial elongation of the arms 174, which may cause axial sliding of one or more of the stents/anchors 171. Alternatively, the stents/anchors 171 may be secured to the blood vessel 61 to prevent axial sliding thereof, whereas cyclical elongation and shortening of the arms 172 due to outward expansion/deflection in inward straightening/narrowing can cause axial compression and expansion of the blood vessel segment 61. For example, the implant 170 may be implanted with the arms 172 initially in the elongated configuration shown in FIG. 27A, wherein the arms 172 may be configured in a biased shape to expand as shown in FIGS. 26A, thereby shortening the axial distance d_{c} of the arms 172 and blood vessel 61 between the anchors/stents 171. That is, the distance d_{c} of the arms 172 and the blood vessel 61 in such segment can cyclically reduce and expand as the arms expand and compress radially. The blood vessel segment 69 may cyclically transition between the oval configuration shown in FIG. 26B and the more circular configuration shown in FIG. 27B, thereby evening-out and/or increasing blood flow through the blood vessel segment 61 during one or more portion(s) of the cardiac cycle, as with other compliance-enhancing implant devices disclosed herein.

Various of the vessel-reshaping implant devices disclosed above include stents having vessel-reshaping arms projecting from, or associated with, only a single axial side of the stent(s), which may be sufficient to produce desired compliance enhancement in some applications. However, it should be understood that examples disclosed herein including vessel-reshaping arms can advantageously include any number or arms, or sets of arms, one or more of which may project/emanate either or both axial ends/sides of an implant device (e.g., of a stent device or other anchoring structure). For example, some examples of the present disclosure include an anchoring structure (e.g., stent) having a first set (i.e., one or more (e.g., two)) of vessel-reshaping arms associated with a first axial end/side of the anchoring structure (or of one anchoring structure of a multi-anchor example) and a second set (i.e., one or more (e.g., two)) of vessel-reshaping arms associated with a second axial end/side of the anchoring structure (or of one anchoring structure of a multi-anchor example). Such implementations can produce a device having an hourglass-shaped side profile in a relaxed state of the implant device.

Described below are various example compliance-enhancing implant devices having two sets of vessel-reshaping arms configured to reversibly alter a target vessel's cross-sectional shape from circular to oval, thereby mimicking natural compliance, as described in detail throughout the present disclosure. Such examples can provide a flow-leveling function by decreasing blood vessel volume, thereby increasing pressure, during diastole and increasing blood vessel volume, thereby decreasing pressure, during systole.

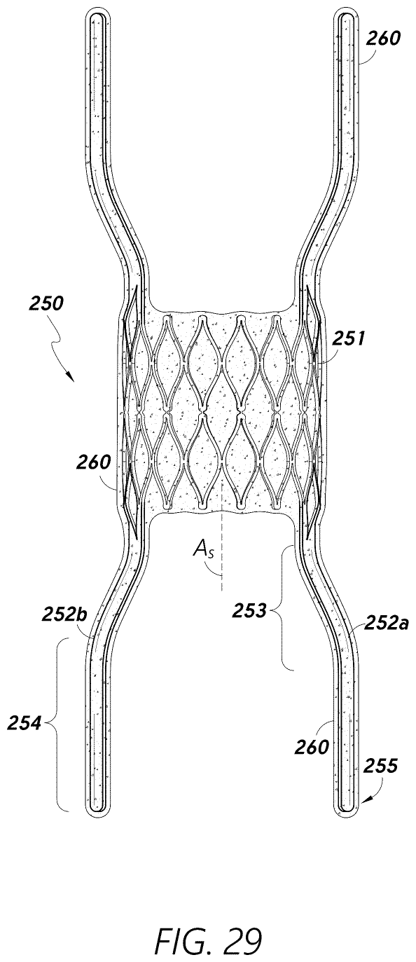

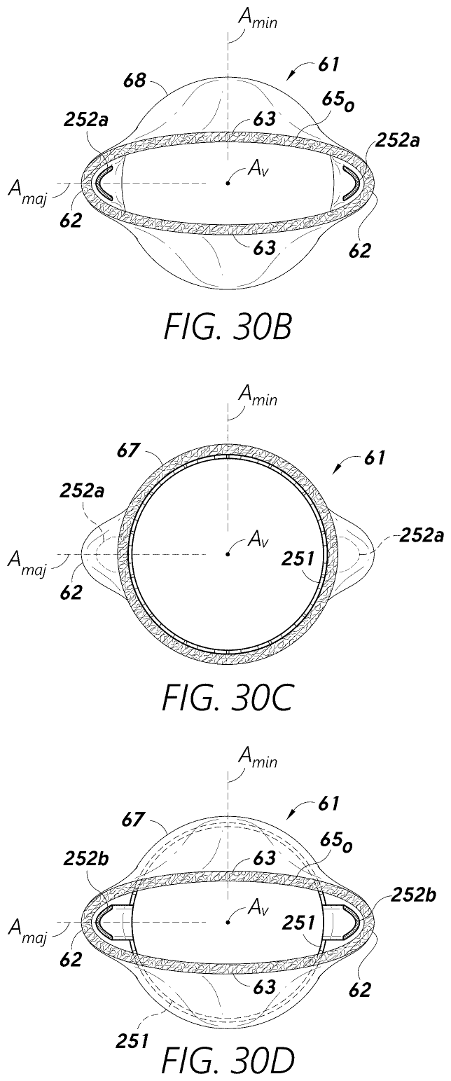

FIGS. 28A and 28B show views of a flow-leveling stent 250 including vessel re-shaping/ovalizing arms 252 in accordance with one or more examples. FIGS. 30A and 30B show side cutaway and axial cross-sectional views, respectively, of the flow-leveling stent 250 implanted in an example blood vessel segment 61. The arms 252 of the implant 250 emanate from, and/or are coupled to, an anchor 251 (e.g., stent segment) configured to be expanded and secured within the blood vessel segment 61 to hold the implant 250 in-place therein. Compared to stent implants including fully-circumferential stent frames configured to form an oval shape to thereby ovalize a target blood vessel segment, the implant 250 and other implants including ovalizing arms that extend axially from a stent or other anchor can be relatively minimalistic, at least with respect to ovalizing segments thereof. Therefore, implants including double-sided ovalizing arms, such as the implant 250, can produce blood vessel re-shaping functionality with reduced structural bulkiness and/or reduced blood flow impedance. Furthermore, with two sets of arms 252, with a set of one or more arms on each axial side of an anchor 251 (or implant assembly), the compliance-enhancement/vessel-reshaping produced relative to the structural bulkiness may be particularly high/effective.

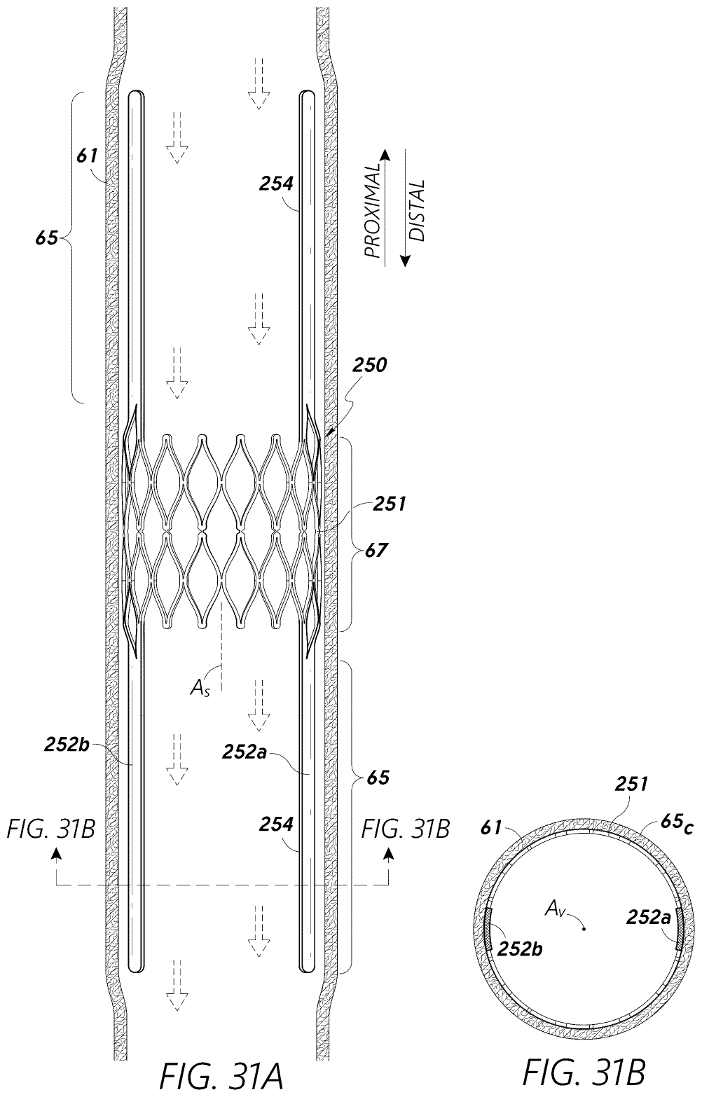

The implant device 250 can include a cylindrical stent 251. Each base/end/side 271 of the cylindrical stent/anchor 251 can include at least two diametrically opposed, outwardly-deflecting and/or curved arms 252. The curved arms 252 can be configured to extend/deflect away from the circular axis Aₛ of the cylindrical stent/anchor 251 before recurving/deflecting slightly toward the circular axis Aₛ in response to blood-pressure-induced force on the arms 252. As shown in the accompanying figures, the two sets of arms 252 can be essentially coplanar, however, other configurations are possible. Each of the arms 252, as with other examples disclosed herein, can be configured to be outwardly biased, as shown in FIG. 28B, but capable of being elastically deformed into a less-curved configuration, as shown in FIG. 31A.

With reference to FIGS. 30A-30D, upon implantation, the outward bias of the arms 252 presses outwardly on the vessel 61 in which the device 250 is implanted. Once implanted, during a low-pressure state (e.g., diastole), portions 65 of the vessel 61 on upstream and downstream sides, respectively, of the anchor/stent 251 and associated vessel portion 67 are deformed from a roughly circular shape to an oval-shaped cross section having reduced cross sectional area. The axial views of FIGS. 30B and 30D show the blood vessel portions 65 ovalized axially outside of the anchor portion 67 by the anchor arms 252. During a high-pressure state (e.g., systole), pressure within the blood vessel lumen 61 increases, forcing the oval-shaped cross section areas 650 to a more circular-shaped cross section 65c (see FIGS. 31A and 31B) and elastically deforming the arms 252 into a less-curved/deflected configuration. As blood pressure conditions cyclically return to the lower-pressure state, the arms 252 of the implant 250 return to their biased, outwardly-deflected configuration. Compared to certain examples comprising vessel-reshaping arms on only one side of an implant anchor/stent, examples comprising sets of reshaping arms on both sides of an anchor/stent can advantageously produce reshaping of a relatively longer blood vessel segment, thereby more closely/effectively mimicking natural blood vessel compliance.

As described and shown, the arms 252 extend/project from both axial ends of the stent/anchor frame 251, as shown. In some implementations, the arms 252 may be an integrated form with the stent frame 251. The arms 252 project in distal and proximal directions from the frame 251. The arms 251 may terminate at distal ends thereof in free ends 255, or the arms on a given side of the implant 250 may be coupled together across the diameter of the implant, as with certain other examples disclosed herein.

The implant arms 252 can be used to leverage the aspect of tubular blood vessels generally having a maximum cross-sectional area and volume for a given perimeter when the cross-sectional area thereof is that of a circle, and therefore deviations from the circular shape produce a decrease in area/volume relative to the circular shape. As described above, to the extent that a target blood vessel is non-compliant, the vessel may have a relatively fixed/inelastic circumference. Therefore, forcing a non-compliant blood vessel into a non-circular cross-sectional shape can result in the volume of blood flowing through the blood vessel being forced through a relatively smaller volume, thereby exerting increased pressure on the blood flow. The implant arms 252, in a biased state thereof, are configured to reshape the blood vessel 61 to a non-circular shape, wherein pressure increase in the blood vessel 61 (e.g., during systole) causes the hoop stress in the blood vessel wall to overcome the biased shape of the arms 252, thereby straightening the arms and allowing the blood vessel to become more circular.

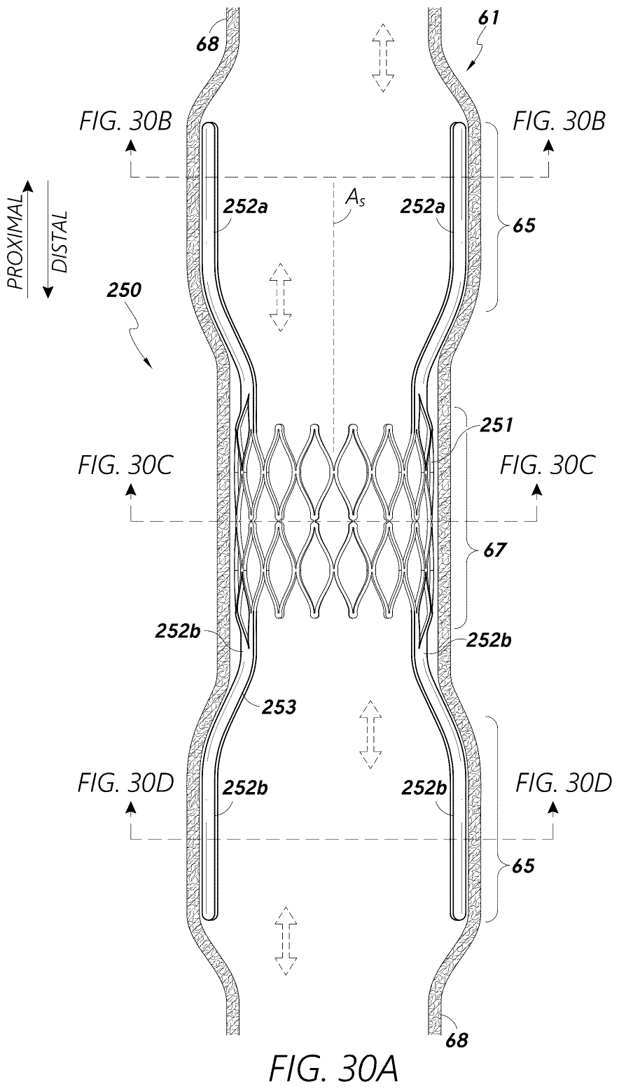

FIG. 29 shows an example implementation of the blood-vessel-reshaping implant device 250, wherein at least a portion of the anchor/stent 251 and/or reshaping arms 252 is covered with a covering 260. Although the illustration of FIG. 29 shows the covering 260 covering an implant 250 having dual-sided reshaping arms 252, it should be understood that any of the example implant devices disclosed herein can comprise coverings as shown and described in connection with FIG. 29. The material of the covering 260 can be chosen to reduce the risk of tissue erosion/abrasion due to pressure and/or friction against the inner blood vessel walls. Additionally or alternatively, the covering 260 can comprise material(s) and/or treatment(s) configured to promote or inhibit tissue overgrowth. As possible examples, the covering 260 can comprise one or more of: cloth, tissue, silicone, polymeric material, and/or other suitable biocompatible material(s). In some implementations, the covering 260 comprises at least one of: expanded polytetrafluoroethylene (ePTFE), polyester, polyurethane, fluoropolymers (e.g., perfluoroelastomers and the like), polytetrafluoroethylene, polyethylene terephthalate (Dacron), silicones, urethanes, ultra-high molecular weight polyethylene, aramid fibers, and combinations thereof.

The covering 260 can be positioned on an inner side/diameter and/or outer side/diameter of the frame 251 and/or arms 252. In some implementations, the covering covers only one of the stent/anchor portion 251 or the arms portion 252 of the implant device 250, but not both.

The stent portion/segment 251 of the implant 250 can include a wire-frame stent or anchor, and may be similar to any of the stent devices disclosed herein. The arms 252 can be positioned on both upstream and downstream sides of the stent 251. The arms 252 may be coupled to the stent/anchors 251 and/or integrated therewith in some manner. For example, the anchor 251 and arms 252 may be cut from a tube, sheet, or other structure, such that the anchor 251 and arms 252 comprise a unitary, integrated form. In some implementations, attachment means, such as clips, sutures, hooks, clamps, or other fasteners, are used to attach the arms 252 to the anchor/stent 251.

The arms 252 can comprise spring projections/extensions from the anchor 251, wherein the arms 252 have an outward bias with respect to the axis Aₛ of the anchor/stent 251. The outward bias of the arms 252, as shown in FIGS. 30A-30D, causes the segments 65 of the vessel 61 spanned by the outwardly-deflected arm portions 252 to take on an oval cross-sectional shape. On a given axial end/side of the anchor 251, one of the arms 252 may advantageously emanate from one circumferential position of the anchor 251, whereas a second one of the arms 252 emanates from a position on an opposite circumferential/diametrical side/portion of the anchor 251, as shown. Although two arms 252 are shown on each axial end/side of the implant 251, it should be understood that more (or less) than two ovalizing arms may extend from each end of the stent/anchor 251. The arms 252 may each terminate in respective free ends 255, which may have certain atraumatic features, such as curved surfaces, or the like. For example, the free ends 255 may deflect/project radially inwardly to avoid scraping/abrading the inner blood vessel wall.

As with other examples disclosed herein, the example implants of FIGS. 28-30 include vessel-reshaping arms 252 that are biased to a wider diameter compared to the diameter of the stent/anchor segment(s) from which the arms emanate. For example, in a relaxed/non-pressurized state, a first diameter dₐ of the arms 252 has a greater dimension compared to a diameter d_{b} of the stent/anchor 251, wherein the arms 252 are configured to transition to a straighter, reduced diameter (e.g., diameter d_{b}, or a diameter between dₐ and d_{b}) when pressure within the blood vessel 61 overcomes the expansion/deflection bias of the arms 252 and allows for the blood vessel segments 65 to revert to a more-circular cylindrical shape.

FIGS. 31A and 31B show side cutaway and axial cross-sectional views, respectively, of the flow-leveling implant 250 with the arms 252 in a straightened configuration in a high-pressure condition in accordance with one or more examples. Though the arms 252 of the implant 250 may be biased toward the outwardly-deflected configuration shown in FIGS. 28-30, they may be configured to, when subjected to mechanical forces associated with high luminal pressure, responsively transform to a more-straightened systolic configuration (see FIGS. 31A, 31B).

During systole, pressure through the oval cross-sectional shape formed by the arms 252 causes pressure to increase, which may force the circumferential area of the vessel farthest from the arms 252 (e.g., in the minor-axis Aₘᵢₙ area of the ovalized blood vessel segments 65) to push outward, overcoming the spring force of the arms 252, and bringing them inward against the spring bias. This in turn causes the vessel 61 to take on a rounder cross-sectional shape through some or all of the length of the implant, thereby creating compliance. When the vessel-reshaping arms 252 are forced to a straighter/narrower configuration (with respect to the axial dimension Aₛ of the anchor 251), energy may be stored in the shape memory of the arms 252, such as in the deflection/transition portions 253 thereof, wherein recoil/expansion of the arms 252 towards their biased, ovalizing/expanded configuration can return/release energy to the blood circulation. When the arms 252 are straightened, they may generally be more in-line/parallel with the axis Aₛ of the anchor/stent 251.

As shown in FIGS. 31A and 31B, luminal pressure forces against the blood vessel wall 61 increase the hoop stress on the blood vessel, which may force the blood vessel, and with it the arms 252, to conform to a more-circular shape. The resulting hoop stress, also referred to as “tangential stress” or “circumferential stress,” from luminal pressure increase exerts radially-outward force along the blood vessel's inner circumference, such stresses/forces being tensile in nature, which can tend to cause the blood vessel to increase in diameter. However, where the elasticity of the blood vessel wall is compromised, as with the blood vessel 61, the expansion of the blood vessel diameter is limited, and therefore the pressure increase reshapes the blood vessel to a more circular shape without substantially increasing the circumference thereof. The blood pressure force on the blood vessel wall and resulting inward deflection of the major-axis blood vessel walls 62 (due to outward deflection of the vessel wall portions 63 in the area of the minor axis Aₘᵢₙ) may cause inward deflection of the arms 252 to form a desired geometric change to conform to a more-circular shape of the anchor/stent 251 and blood vessel 61.

With respect to implantation within the aorta or other arterial blood vessel, the systolic phase of the cardiac cycle, during which pressure levels in the aorta/arteries are relatively higher, causes the expansion of the blood vessel 61 and inward deflection of the arms 252 to conform to the more-circular shape (shown in FIG. 31B), whereas the diastolic phase, which is associated with relatively lower arterial blood pressure levels, allows for the expansion of the arms 252 in the major axis Aₘₐⱼ dimension to the lower-energy ovalizing configuration, thereby forcing the blood vessel 61 to assume a more oval shape.

With reference to the axial view of FIG. 31B, as the spring arms 252 on both axial ends/sides of the anchor 251 push outward on the major-axis walls/portions 62 of the blood vessel 61, the minor-axis walls/portions 63 of the blood vessel 61 may be pulled/drawn at least partially towards an axial center Aᵥ of the blood vessel 61 and/or towards each other in a manner as to cause the blood vessel 61 to form a non-circular/non-cylindrical shape, such as an oval shape as shown in FIGS. 30B-30D. That is, compared to the circular/cylindrical shape of the blood vessel 61, the blood vessel 61 in the oval shape has a cross-sectional area that is reduced in the axial segments/areas 65 where the deflected portions 254 of the arms 252 are implanted.

As the pressure in the blood vessel 61 increases (e.g., in connection with the systolic phase of the cardiac cycle), the plastically-deformable nature of the spring arms 252 allows for the arms 252 to be deformed/remodeled to the straighter and/or narrower configuration to accommodate the shortening of the major axis dimension Aₘₐⱼ of the blood vessel segments 65. The implant arms 252 improve cardiac perfusion by causing a decrease in cross-sectional blood vessel area during diastole relative to systole due to the reshaping of the blood vessel 61 caused by the lengthening of the major diameter Aₘₐⱼ of the blood vessel 61.

The anchor/stent 251 and/or arms 252 may be inclined to experience tissue in-growth in one or more areas thereof, which may or may not be facilitated by a covering or similar feature of the implant 250. As with any of the examples disclosed herein, the implant arms 252 can be configured to deform/transition from the ovalizing outwardly-deflected configuration of FIGS. 28-30 to the more-circular shape of FIGS. 31A and 31B in the presence of threshold blood pressure levels greater than 80 mmHg, such as blood pressure levels greater than 90 mmHg (e.g., between 90-120 mmHg).

In some examples, the multi-stent/anchor example of FIGS. 26-27, comprising multiple stents coupled by connecting intermediate arms (see arms 172 in FIGS. 26A and 27A) can be combined with features of examples including one or more sets of free arms extending in distal and/or proximal directions as in FIGS. 28-31. That is, examples of the present disclosure can include both anchor/shunt-to-anchor/shunt connecting ovalizing/reshaping arms as well as distal and/or proximal ovalizing/reshaping arms extending from anchor/stent end(s) and having free ends. Such examples can advantageously provide increased vessel reshaping.

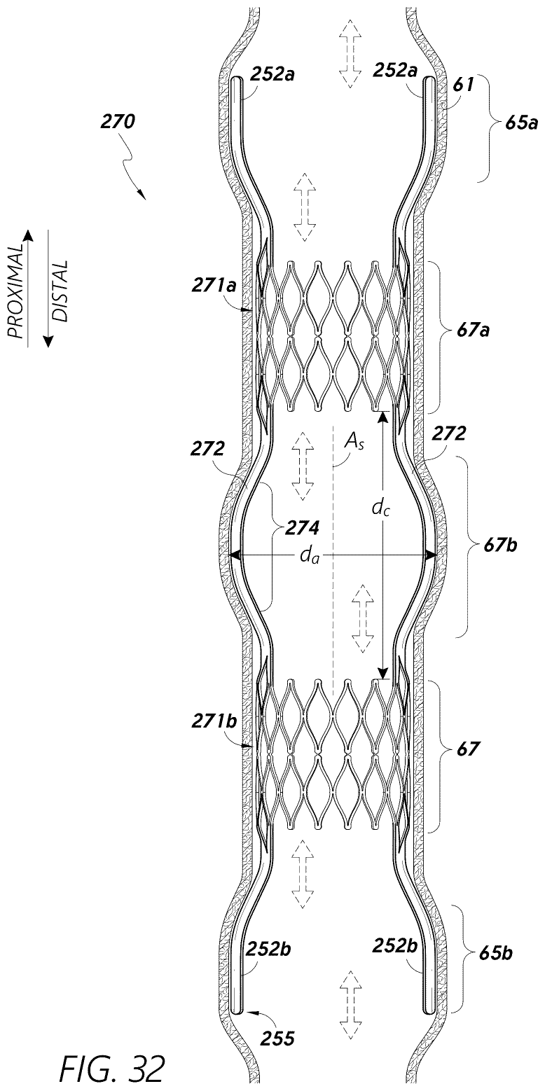

FIG. 32 shows a side view of a blood vessel reshaping implant assembly/device 270 comprising a plurality of anchors (e.g., circular cylindrical stents) 271 coupled by vessel reshaping arm(s) 272, wherein the anchors 271 further have axially-projecting free reshaping arms 252 associated therewith, as disclosed in one or more examples herein. The implant 270 may have any feature(s) of any of the other examples of implant devices including blood-vessel-ovalizing/reshaping arms disclosed herein, in combination and/or individually.

The implant 270 may advantageously include multiple stent segments 271, such as the illustrated stent segments 271a and 271b, wherein the stent/anchor segments 271 may be axially offset from one another and connected by connector arms 272. For example, two or more anchor arms 272 may span the axial distance d_{c} between the stent/anchor segments 271a, 271b. In the illustrated example, two arms 272 extend between opposite sides of the stents/anchors 271 (e.g., from the distal side of the proximal anchor 271a and the proximal side of the distal anchor 271b). The arms 272 may deflect radially outwardly, as with other examples disclosed herein. For example, at least an axial segment 274 of the arms may produce an expanded ovalizing major-axis diameter dₐ, which in turn produces an expanded major-axis dimension of the blood vessel segment 69 in which the expanded arm portions 274 are disposed.

Each of the anchors/stents 271, on an axially-outside base/end/side thereof, can include a set (e.g., one or more) of outwardly-deflecting and/or curved arms 252, which may advantageously be diametrically opposed on a given side/end of the implant 270. The curved arms 252 can be configured to extend/deflect away from the axis Aₛ of the stent(s)/anchor(s) 271 before recurving/deflecting slightly toward the circular axis Aₛ in response to blood-pressure-induced force on the arms 252. The vessel reshaping arms 272, 252 may be configured like any other example reshaping arms disclosed herein. Each of the arms 272, 252, as with other examples disclosed herein, can be configured to be outwardly biased, as shown in FIG. 32, but capable of being elastically deformed into a straighter configuration (e.g., more-parallel with the axis Aₛ) when deforming forces act thereon. As described and shown, the arms 272, 252 may be an integrated form with the stent frames 271. The arms 252 may terminate at distal ends thereof in free ends 255. Alternatively, the arms 252a may be coupled together across the diameter of the implant 270, as with certain other examples described herein (and/or the arms 252b may be coupled together).

The anchors 271 can comprise cylindrical stents, as with other examples disclosed and shown in detail in the present disclosure. However, it should be understood that any of the stent anchors disclosed herein may have a biased oval/non-circular shape, such that the stents are configured to ovalize the blood vessel segments in which they are implanted, along with the ovalizing/reshaping effect/functionality of any reshaping arms associated with the respective example. For example, with respect to the example of FIG. 32, the anchors 271 may have a biased non-circular (e.g., oval) shape configured to cause reshaping of the blood vessel segments 67a, 67b to some degree in relatively low-pressure conditions.

With further reference to the implant 270 shown in FIG. 32, in high pressure conditions, the arms 252, 272 may straightened-out, thereby allowing the blood vessel 61 to assume a more-circular configuration in the blood vessel segments 65a, 65b, 69. The straightening/narrowing of the arms 252, 272 may cause some amount of axial elongation of the arms 272, which may cause axial sliding of one or more of the stents/anchors 271. Alternatively, the stents/anchors 271 may be secured to the blood vessel 61 to prevent axial sliding thereof, whereas cyclical elongation and shortening of the arms 272 due to outward expansion/deflection in inward straightening/narrowing can cause axial compression and expansion of the blood vessel segment 61. The blood vessel segments 65a, 65b, and 69 may cyclically transition between an oval configuration and a more-circular configuration, thereby evening-out and/or increasing blood flow through the blood vessel segment 61 during one or more portion(s) of the cardiac cycle, as with other compliance-enhancing implant devices disclosed herein.

### Additional Description of Examples

Provided below is a list of examples, each of which may include aspects of any of the other examples disclosed herein. Furthermore, aspects of any example described above may be implemented in any of the numbered examples provided below.

Example 1: An implant device comprising an elongated stent structure forming an axial flow channel therethrough, wherein the stent structure has a shape-memory that biases the stent structure to a bent configuration, such that the stent structure is configured to return to the bent configuration after manipulation to a straightened configuration of the stent structure.

Example 2: The implant device of any example herein, in particular example 1, wherein the stent structure comprises a first anchor coupled to a second anchor by a connecting arm that is biased in a bent configuration.

Example 3: The implant device of any example herein, in particular example 2, wherein the first anchor and the second anchor are stents.

Example 4: The implant device of any example herein, in particular example 3, wherein the first anchor and the second anchor are tapered towards the connecting arm.

Example 5: The implant device of any example herein, in particular any of example 2 or example 3, wherein the first anchor and the second anchor comprises a C-shaped anchor.

Example 6: The implant device of any example herein, in particular example 2, wherein at least one of the first anchor and the second anchor comprises a contact pad.

Example 7: The implant device of any of any example herein, in particular any of examples 1-6, wherein, in the bent configuration, an axis associated with an inlet end of the stent structure is angled at a bend angle relative to an axis associated with an outlet end of the stent structure.

Example 8: The implant device of any example herein, in particular example 7, wherein the bend angle is an acute angle.

Example 9: The implant device of any example herein, in particular example 7, wherein the bend angle is approximately 90°.

Example 10: The implant device of any example herein, in particular example 7, wherein the bend angle is between 90°-120°.

Example 11: The implant device of any example herein, in particular example 7, wherein the bend angle is formed by a discrete bend at a lengthwise medial portion of the stent structure.

Example 12: The implant device of any example herein, in particular example 11, wherein bend is at a lengthwise center of the stent structure.

Example 13: The implant device of any example herein, in particular example 7, wherein the bend angle is formed by a gradual bend over a length of the stent structure.

Example 14: The implant device of any of any example herein, in particular any of examples 1-13, wherein, in the bent configuration, the stent structure has a non-circular axial cross-sectional shape.

Example 15: The implant device of any example herein, in particular example 14, wherein, in the straightened configuration, the stent structure has a circular axial cross-sectional shape.

Example 16: The implant device of any of any example herein, in particular any of examples 1-15, wherein the stent structure has an internal fluid-tight covering.

Example 17: The implant device of any of any example herein, in particular any of examples 1-16, wherein the stent structure has an external fluid-tight covering.

Example 18: The implant device of any of any example herein, in particular any of examples 1-17, wherein the implant device is sterilized.

Example 19: A method of managing flow in a blood vessel, the method comprising accessing a target site in a blood vessel using a transcatheter access path, deploying a stent at the target site in the blood vessel, reshaping the blood vessel to an axially-bent configuration of the blood vessel by causing the stent to assume a biased, axially-bent configuration of the stent within the blood vessel, and reshaping the blood vessel to a relatively-straight configuration of the blood vessel using pressure forces of blood disposed within the blood vessel.

Example 20: The method of any example herein, in particular example 19, wherein said causing is performed automatically by shape-memory material of the stent.

Example 21: The method of any example herein, in particular any of example 19 or example 20, wherein said reshaping the blood vessel to the relatively-straight configuration of the blood vessel involves overcoming a shape-memory bias in the stent to reshape the stent to a relatively-straight configuration of the stent.

Example 22: The method of any example herein, in particular example 21, wherein said reshaping the blood vessel to the relatively-straight configuration of the blood vessel is caused at least in part by said reshaping the stent to the relatively-straight configuration of the stent.

Example 23: The method of any example herein, in particular example 22, wherein the stent comprises a fluid-tight covering.

Example 24: The method of any example herein, in particular example 23, wherein said reshaping the stent to the relatively-straight configuration of the stent is caused at least in part by fluid pressure forces against the covering.

Example 25: The method of any of any example herein, in particular any of examples 21-24, wherein said reshaping the stent to the relatively-straight configuration of the stent is caused at least in part by said reshaping of the blood vessel to the relatively-straight configuration of the blood vessel.

Example 26: The method of any example herein, in particular example 25, wherein said reshaping of the blood vessel to the relatively-straight configuration of the blood vessel is caused at least in part by application of fluid pressure forces through open cells of the stent against an internal wall of the blood vessel.

Example 27: The method of any of any example herein, in particular any of examples 19-26, further comprising, subsequent to said reshaping the blood vessel to the relatively-straight configuration of the blood vessel, returning the blood vessel to the axially-bent configuration of the blood vessel by returning the stent to the axially-bent configuration of the stent within the blood vessel.

Example 28: The method of any example herein, in particular example 27, wherein said returning the blood vessel to the axially-bent configuration increases at least one of blood flow or blood pressure downstream of the stent.

Example 29: The method of any of any example herein, in particular any of examples 19-28, wherein said reshaping the blood vessel to the relatively-straight configuration of the blood vessel causes mechanical energy to be stored in the stent.

Example 30: The method of any example herein, in particular example 29, wherein such mechanical energy comprises spring forces of struts of the stent associated with a shape memory of the struts.

Example 31: The method of any of any example herein, in particular any of examples 19-30, wherein said reshaping the blood vessel to the relatively-straight configuration of the blood vessel tempers at least one of a pressure or a flow of blood within the target site in the blood vessel.

Example 32: The method of any of any example herein, in particular any of examples 19-31, wherein said reshaping the blood vessel to the axially-bent configuration of the blood vessel involves reshaping an axial cross-section of the blood vessel to a non-circular shape.

Example 33: The method of any example herein, in particular example 32, wherein said causing the stent to assume the axially-bent configuration of the stent involves causing the stent to assume a non-circular cross-sectional shape with respect to one or more portions thereof.

Example 34: The method of any example herein, in particular example 33, further comprising causing reshaping the stent from the non-circular cross-sectional shape to a more-circular cross-sectional shape when the blood vessel is reshaped to the relatively-straight configuration.

Example 35: The method of any example herein, in particular example 32, wherein said reshaping the blood vessel to the relatively-straight configuration of the blood vessel involves reshaping the axial cross-section of the blood vessel to a more-circular shape from the non-circular shape.

Example 36: The method of any of any example herein, in particular any of examples 19-35, further comprising sanitizing the stent.

Example 37: An implant device comprising a first arm having a first tissue-contact surface, a second arm having a second tissue-contact surface, and a bend portion coupling the first arm to the second arm, the bend portion being spring-biased to hold the first arm and the second arm in a crimped configuration.

Example 38: The implant device of any example herein, in particular example 37, wherein a side profile of the implant device has a horseshoe shape.

Example 39: The implant device of any example herein, in particular any of example 37 or example 38, wherein in the crimped configuration, the first arm and the second arm are positioned such that the first tissue-contact surface and the second tissue-contact surface are a first distance apart, and application of force to pull the first arm and the second arm apart such that the first tissue-contact surface and the second tissue-contact surface are a second distance apart that is greater than the first distance causes the bend portion to exert force on at least one of the first arm or the second arm to urge the first arm and the second arm closer together.

Example 40: The implant device of any example herein, in particular example 37, wherein application of force to pull the first arm and the second arm apart, thereby increasing a separation distance between the first tissue-contact surface and the second tissue-contact surface, causes the bend portion to store spring energy that urges the first and second arms together.

Example 41: The implant device of any example herein, in particular example 40, wherein the separation distance between the first tissue-contact surface and the second tissue-contact surface increases moving towards the bend portion.

Example 42: The implant device of any of any example herein, in particular any of examples 37-41, wherein the first arm and the second arm are outwardly-concave with respect to a center of the implant device between the first arm and the second arm.

Example 43: The implant device of any example herein, in particular example 42, wherein the center of the implant device corresponds to a center of curvature of the bend portion.

Example 44: The implant device of any of any example herein, in particular any of examples 37-43, wherein the first arm, the second arm, and the bend portion are formed of a unitary form.

Example 45: The implant device of any example herein, in particular example 44, wherein the unitary form has a curved inner surface, the inner surface comprising the first tissue-contact surface and the second tissue-contact surface.

Example 46: The implant device of any of any example herein, in particular any of examples 37-45, wherein the implant device is configured to be implanted in an aorta of a patient such that the first arm is disposed at least partially within an ascending aorta, and the second arm is disposed at least partially within a descending aorta.

Example 47: The implant device of any of any example herein, in particular any of examples 37-46, wherein the implant device is sterilized.

Example 48: A method of managing flow in an aorta, the method comprising accessing, with a delivery system holding a clamp device, an ascending portion of an aorta using a transcatheter access path, deploying a distal arm of the clamp device from the delivery system in the ascending portion of the aorta, proximally withdrawing the delivery system through an arch portion of the aorta, and deploying a proximal arm of the clamp device in a descending portion of the aorta.

Example 49: The method of any example herein, in particular example 48, further comprising crimping the arch portion of the aorta by applying force to at least one of the distal arm or the proximal arm.

Example 50: The method of any example herein, in particular any of example 48 or example 49, further comprising deploying a bend portion of the clamp device in the arch portion of the aorta.

Example 51: The method of any example herein, in particular example 50, wherein the bend portion is positioned at a medial portion of the clamp device between the distal arm and the proximal arm.

Example 52: The method of any example herein, in particular any of example 50 or example 51, wherein the bend portion is spring-biased to hold the distal arm and the proximal arm at a pre-set separation distance.

Example 53: The method of any example herein, in particular example 52, wherein the bend portion is configured to apply a force to at least one of the distal arm or the proximal arm urging the distal arm and the proximal arm closer together when the distal arm and the proximal arm are displaced by a distance that is greater than the pre-set separation distance.

Example 54: The method of any of any example herein, in particular any of examples 48-53, further comprising exposing the clamp device to systolic blood pressure forces within the aorta, wherein the systolic blood pressure forces cause the aorta to un-crimp in a manner as to pull the distal arm and the proximal arm away from one another, thereby storing energy in a spring-biased bend portion of the clamp device.

Example 55: The method of any example herein, in particular example 54, further comprising, subsequent to said exposing the clamp device to systolic blood pressure forces within the aorta, exposing the clamp device to diastolic blood pressure forces within the aorta, wherein the diastolic blood pressure forces permit the spring-biased bend portion to use the energy to pull the distal arm and the proximal arm towards one another to crimp the arch portion of the aorta.

Example 56: The method of any of any example herein, in particular any of examples 48-55, further comprising sanitizing the clamp device prior to said accessing the ascending portion of the aorta.

Example 57: An implant device comprising a first stent anchor configured to be secured within a blood vessel, and first and second arms extending from an axial end portion of the first stent anchor, the first and second arms deflecting to an expanded diameter that is greater than a diameter of the first stent anchor.

Example 58: The implant device of any example herein, in particular example 57, wherein the first stent anchor and the first and second arms comprise an integrated form.

Example 59: The implant device of any example herein, in particular example 57 or example 58, wherein the first and second arms project from a frame of the first stent anchor.

Example 60: The implant device of any example herein, in particular any of examples 57-59, wherein the first and second arms terminate at respective free ends.

Example 61: The implant device of any example herein, in particular any of examples 57-60, wherein the first and second arms are joined at a distal portion of the implant device.

Example 62: The implant device of any example herein, in particular example 61, wherein the first and second arms are joined by a crossbar that spans the diameter of the first stent anchor.

Example 63: The implant device of any example herein, in particular example 62, wherein the first and second arms and the crossbar are formed by a single bar that forms a closed loop with the first stent anchor.

Example 64: The implant device of any example herein, in particular example 57, wherein the first stent anchor has a circular cross-sectional shape.

Example 65: The implant device of any example herein, in particular example 57, wherein the first stent anchor has an oval cross-sectional shape.

Example 66: The implant device of any of any example herein, in particular any of examples 57-65, further comprising a second stent anchor coupled to the first and second arms at a first axial end of the second stent anchor.

Example 67: The implant device of any example herein, in particular example 66, wherein the first and second arms hold the second stent anchor at an axially-offset position relative to the first stent anchor.

Example 68: The implant device of any example herein, in particular example 67, further comprising a first set of radially-deflected arms extending from a second axial end portion of the first stent anchor, and a second set of radially-deflected arms extending from a second axial end portion of the second stent anchor.

Example 69: The implant device of any example herein, in particular example 68, wherein the first set of radially-deflected arms and the second set of radially-deflected arms have free ends.

Example 70: The implant device of any example herein, in particular example 57, further comprising third and fourth arms extending from a second axial end portion of the first stent anchor, the third and fourth arms deflecting to a second expanded diameter that is greater than the diameter of the first stent anchor.

Example 71: The implant device of any example herein, in particular example 70, wherein the first expanded diameter and the second expanded diameter are the same.

Example 72: The implant device of any of any example herein, in particular any of examples 57-71, wherein at least a portion of at least one of the first stent anchor, first arm, or second arm is covered with a covering.

Example 73: The implant device of any example herein, in particular example 72, wherein the covering is configured to promote tissue overgrowth over at least a portion of the implant device.

Example 74: The implant device of any example herein, in particular example 72, wherein the covering is configured to impede tissue overgrowth.

Example 75: A method of managing flow in an aorta, the method comprising accessing, with a delivery system holding a stent implant device, a target blood vessel using a transcatheter access path, the stent implant device including first and second arms projecting axially from a first axial end of the stent frame, deploying the first stent frame in a first segment of the target blood vessel, and ovalizing a second segment of the target blood vessel on a first axial side of the stent frame using the first and second arms.

Example 76: The method of any example herein, in particular example 75, wherein said ovalizing the second segment of the target blood vessel involves deflecting the first and second arms radially outward with respect to an axis of the first stent frame.

Example 77: The method of any example herein, in particular example 75 or example 76, further comprising allowing the target blood vessel to deflect the first and second arms radially inward to circularize the second segment of the target blood vessel.

Example 78: The method of any example herein, in particular example 77, wherein said deflecting the first and second arms radially inward is caused at least in part by increased blood pressure within the target blood vessel.

Example 79: The method of any example herein, in particular example 77 or example 78, wherein said deflecting the first and second arms radially inward causes the first and second arms to straighten.

Example 80: The method of any of any example herein, in particular any of examples 75-79, wherein the first and second arms are coupled by a distal crossbar.

Example 81: The method of any example herein, in particular example 80, further comprising allowing the target blood vessel to deflect the first and second arms radially inward, thereby causing the crossbar to bow axially outward.

Example 82: The method of any of any example herein, in particular any of examples 75-81, further comprising deploying a second stent frame in a third segment of the target blood vessel, the second segment being disposed between the first segment and the third segment.

Example 83: The method of any example herein, in particular example 82, wherein the second stent frame is coupled to the first and second arms.

Example 84: The method of any example herein, in particular example 82 or example 83, further comprising axially shortening the target blood vessel by radially-outwardly deflecting the first and second arms.

Example 85: The method of any example herein, in particular example 75, wherein the stent implant device further includes third and fourth arms projecting axially from a second axial end of the stent frame, and the method further comprises ovalizing a third segment of the target blood on a second axial side of the stent frame using the third and fourth arms.

Example 86: The method of any example herein, in particular example 85, wherein the first axial side is an upstream side of the stent frame and the second axial side is a downstream side of the stent frame.

Example 87: The method of any example herein, in particular example 85, further comprising deflecting the first, second, third, and fourth arms radially inwardly using the target blood vessel.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising.” “including,” “having.” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example. Figure, or description thereof for the purpose of streamlining the disclosure and aiding in the understanding of one or more of the various inventive aspects. This method of disclosure, however, is not to be interpreted as reflecting an intention that any claim requires more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method of increasing compliance in a blood vessel, the method comprising:
accessing a target site in a blood vessel using a transcatheter access path;
deploying a stent at the target site in the blood vessel;
reshaping the blood vessel to an axially-bent configuration of the blood vessel by causing the stent to assume a biased, axially-bent configuration of the stent within the blood vessel, thereby pushing blood through the blood vessel; and
reshaping the blood vessel to a relatively-straight configuration of the blood vessel using pressure forces of blood disposed within the blood vessel.

2. The method of claim 1, wherein said causing is performed automatically by shape-memory material of the stent.

3. The method of claim 1, wherein said reshaping the blood vessel to the relatively-straight configuration of the blood vessel involves overcoming a shape-memory bias in the stent to reshape the stent to a relatively-straight configuration of the stent.

4. The method of claim 1, wherein the stent comprises a fluid-tight covering.

5. The method of claim 4, wherein said reshaping the stent to the relatively-straight configuration of the stent is caused at least in part by fluid pressure forces against the fluid-tight covering.

6. The method of claim 1, wherein said reshaping of the blood vessel to the relatively-straight configuration of the blood vessel is caused at least in part by application of fluid pressure forces through open cells of the stent against an internal wall of the blood vessel.

7. The method of claim 1, further comprising, subsequent to said reshaping the blood vessel to the relatively-straight configuration of the blood vessel, returning the blood vessel to the axially-bent configuration of the blood vessel by returning the stent to the axially-bent configuration of the stent within the blood vessel.

8. The method of claim 1, wherein said reshaping the blood vessel to the axially-bent configuration of the blood vessel involves reshaping an axial cross-section of the blood vessel to a non-circular shape by causing the stent to assume a non-circular cross-sectional shape with respect to one or more portions thereof.

9. The method of claim 1, wherein said reshaping the blood vessel to the relatively-straight configuration increases a volume of blood disposed within a lumen of the stent.

10. A method of increasing compliance in a blood vessel, the method comprising:
accessing a target site in an aorta of a patient using a transcatheter access path;
deploying a stent implant device at the target site in the aorta;
reshaping the blood vessel to an axially-bent configuration of the blood vessel by causing the stent implant device to assume a biased, axially-bent configuration of the stent implant device within the blood vessel, thereby pushing blood through the blood vessel; and
reshaping the blood vessel to a relatively-straight configuration of the blood vessel using pressure forces of blood disposed within the blood vessel.

11. The method of claim 10, wherein said accessing the target site in the aorta involves accessing an inferior vena cava of the patient and crossing over to the aorta through a first wall of the inferior vena cava and a second wall of the aorta to access an inner lumen of the aorta.

12. The method of claim 10, wherein said deploying the stent implant device at the target site in the aorta comprises:
deploying a first anchor in an ascending aorta blood vessel segment adjacent to an aortic valve;
withdrawing a delivery system through an aortic arch segment to deploy a clamp arm along an inner arch portion of the aortic arch segment, the clamp arm being biased to a crimped configuration; and
deploying a second anchor in a descending aorta blood vessel segment.

13. The method of claim 12, wherein an outer arch portion of the aortic arch segment is not covered by the stent implant device.

14. The method of claim 10, wherein the stent implant device comprises a first stent anchor coupled to a second stent anchor by a connecting arm that is biased in a bent configuration.

15. The method of claim 14, wherein the first stent anchor and the second stent anchor are each tapered towards the connecting arm.

16. The method of claim 14, wherein at least one of the first stent anchor and the second stent anchor comprises a C-shaped anchor.

17. The method of claim 14, wherein, in the bent configuration, an axis associated with the first stent anchor is angled at an acute bend angle relative to an axis associated with the second stent anchor.

18. A method of increasing compliance in a blood vessel, the method comprising:
accessing a target site in an aorta of a patient using a transcatheter access path;
deploying a stent at the target site in the blood vessel, the stent having a bias towards an axially-bent shape;
during a diastolic cardiac phase, reshaping the blood vessel to an axially-bent configuration of the blood vessel by allowing the stent to assume its biased, axially-bent shape, thereby increasing blood flow in a blood vessel segment downstream of the stent; and
during a systolic cardiac phase, straightening the stent using a wall of the blood vessel when a pressure in the blood vessel is sufficient to overcome the bias of the stent.

19. The method of claim 18, wherein:
the stent comprises a first stent anchor coupled to a second stent anchor via an elongate arm; and
the method further comprises bending the elongate arm to form an angular offset between an axis of the first stent anchor and an axis of the second stent anchor.

20. The method of claim 18, wherein:
reshaping the blood vessel to the axially-bent configuration involves transitioning a portion of the blood vessel from a circular cross-sectional shape to an oval cross-sectional shape in an area overlapping the stent; and
straightening the stent involves transitioning the portion of the blood vessel from the oval cross-sectional shape back to the circular cross-sectional shape.
